(19) United States
(12) Patent Application Publication
(10) Pub. No.: US 2024/0423547 A1
(43) Pub. Date: Dec. 26, 2024

(54) BLOOD PRESSURE ESTIMATION METHOD AND BIOLOGICAL INFORMATION MEASUREMENT SYSTEM

(71) Applicant: Murata Manufacturing Co., Ltd., Nagaokakyo-shi (JP)

(72) Inventor: Toru SHIMUTA, Nagaokakyo-shi (JP)

(21) Appl. No.: 18/823,762

(22) Filed: Sep. 4, 2024

Related U.S. Application Data
(63) Continuation of application No. PCT/JP2023/009624, filed on Mar. 13, 2023.

(30) Foreign Application Priority Data
Apr. 5, 2022 (JP) 2022-063117
Aug. 12, 2022 (JP) 2022-128972

(51) Int. Cl. A61B 5/00; A61B 5/021; G16H 50/30

(52) U.S. Cl. CPC A61B 5/7239; A61B 5/0059; A61B 5/02116; A61B 5/02125; A61B 5/4809; A61B 5/6826; G16H 50/30; A61B 2560/045; A61B 2560/0462; A61B 2562/0219; A61B 2562/0233

(57) ABSTRACT

A blood pressure estimation method and a biological information measurement system for accurately estimating blood pressure information of a user in a non-invasive manner. A biological information measurement system executes acquiring a photoplethysmographic signal of a blood vessel of a periphery of a user who is a subject by a photoplethysmographic sensor, calculating a peripheral blood pressure index that is an index of a magnitude of a blood pressure of a capillary or an arteriole of the periphery based on a steepness of rising of the photoplethysmographic signal, and estimating a magnitude of a blood pressure of the user by using a de time and the peripheral blood pressure index. The de time is a peak time difference between a d wave and an e wave in an acceleration pulse wave signal obtained by performing second-order differentiation on the photoplethysmographic signal.

### CROSS REFERENCE TO RELATED APPLICATIONS

This application is a continuation of International Application No. PCT/JP2023/009624, filed Mar. 13, 2023, which claims priority to Japanese Patent Application No. 2022-063117, filed Apr. 5, 2022, and Japanese Patent Application No. 2022-128972, filed Aug. 12, 2022, the entire contents of each of which are hereby incorporated in their entirety.

### TECHNICAL FIELD

The present disclosure is directed to a blood pressure estimation method and a biological information measurement system for estimating a blood pressure of a subject (user).

### BACKGROUND

As an index used to estimate a health state of a user, a pulse wave propagating in an artery of the user is used. The pulse wave changes in accordance with a change in the blood pressure of the user at a measurement point. International Publication No. 2015/098977 (the “1977 Publication”), the entire contents of which is hereby incorporated in its entirety, discloses a pulse wave measurement device for measuring a blood pressure with a small burden on a living body. In the pulse wave measurement device disclosed the '977 Publication, blood pressure information of the living body is estimated based on a pulse rate of the living body and time information of the pulse wave of the living body.

However, the estimation of the blood pressure information in the pulse wave measurement device disclosed in the '977 Publication uses the pulse rate of the living body. There is not a high correlation between the pulse rate of the living body and the blood pressure. Therefore, it may not be possible to estimate the blood pressure information in the pulse wave measurement device disclosed in the '977 Publication with high accuracy.

### SUMMARY OF INVENTION

Accordingly, it is an object of the present disclosure to provide a blood pressure estimation method and a biological information measurement system configured for estimating blood pressure information of a subject with high accuracy in a non-invasive manner.

In an exemplary aspect, the present disclosure provides a blood pressure estimation method is configured that includes: acquiring a photoplethysmographic signal of a blood vessel of a periphery of a subject with a photoplethysmographic sensor; calculating a peripheral blood pressure index that is an index of a magnitude of a blood pressure of a capillary or an arteriole of the periphery based on a steepness of rising of the photoplethysmographic signal; and estimating a magnitude of a blood pressure of the subject by using a de time and the peripheral blood pressure index, the de time being a peak time difference between a d wave and an e wave in an acceleration pulse wave signal obtained by performing second-order differentiation on the photoplethysmographic signal. In this aspect, the steps are executed by a biological information measurement system. In addition, a biological information measurement system is provided that includes: a sensing device including a photoplethysmographic sensor configured to acquire a photoplethysmographic signal of a blood vessel of a periphery of a subject; and a signal processing device configured to calculate a peripheral blood pressure index that is an index of a magnitude of a blood pressure of a capillary or an arteriole of the periphery based on a steepness of rising of the photoplethysmographic signal, and to estimate a magnitude of a blood pressure of the subject by using a de time and the peripheral blood pressure index, the de time being a peak time difference between a d wave and an e wave in an acceleration pulse wave signal obtained by performing second-order differentiation on the photoplethysmographic signal.

According to these configurations, the photoplethysmographic signal of the capillary or the arteriole of the periphery of the subject is acquired by the photoplethysmographic sensor, and the peripheral blood pressure index that is the index of the magnitude of the blood pressure of the capillary or the arteriole of the periphery of the subject is calculated based on the steepness of the rising of the acquired photoplethysmographic signal. The magnitude of the blood pressure of the subject is estimated by using the calculated peripheral blood pressure index and the de time in the acceleration pulse wave signal obtained by performing the second-order differentiation on the photoplethysmographic signal. Each of the peripheral blood pressure index and the de time used to estimate the blood pressure has a strong correlation with the blood pressure.

Therefore, according to the present disclosure, a blood pressure estimation method and a biological information measurement system are provided for estimating blood pressure information of a subject with high accuracy in a non-invasive manner.

### BRIEF DESCRIPTION OF DRAWINGS

In the descriptions that follow, like parts are marked throughout the specification and drawings with the same numerals, respectively. The drawings are not necessarily drawn to scale and certain drawings may be illustrated in exaggerated or generalized form in the interest of clarity and conciseness. The disclosure itself, however, as well as a mode of use, further features and advances thereof, will be understood by reference to the following detailed description of illustrative implementations of the disclosure when read in conjunction with reference to the accompanying drawings, wherein:

FIG. 1 is an explanatory diagram illustrating a configuration of a biological information measurement system in accordance with aspects of the present disclosure;

FIG. 2 is an explanatory diagram illustrating an external configuration of a sensing device in accordance with aspects of the present disclosure;

FIG. 3 is an explanatory diagram illustrating an example of a posture of a user when biological information is measured in accordance with aspects of the present disclosure;

FIG. 4 is an explanatory diagram schematically illustrating acquisition of a photoplethysmographic signal by the sensing device in accordance with aspects of the present disclosure;

FIG. 5 is a graph for explaining a maximum amplitude value of the photoplethysmographic signal in accordance with aspects of the present disclosure;

FIG. 6 is a graph for explaining each waveform element required for calculation of a pulse wave feature amount that is a peripheral blood pressure index in accordance with aspects of the present disclosure;

FIG. 7 is a second graph for explaining each waveform element required for the calculation of the pulse wave feature amount that is the peripheral blood pressure index in accordance with aspects of the present disclosure;

FIGS. 8(a) to 8(c) are a set of graphs illustrating a correlation relationship between a steepness of rising of the photoplethysmographic signal and each pulse wave feature amount in accordance with aspects of the present disclosure;

FIGS. 9(a) to 9(f) are a set of graphs illustrating a result of calculating a relationship between a systolic blood pressure and each of pulse wave feature amounts 1/VE0.5, a/S, and (a-b)/(a-d) when a height of a measurement site is changed and when a vicinity of the measurement site is cooled, from each photoplethysmographic signal measured with green light and near-infrared light in accordance with aspects of the present disclosure;

FIGS. 10(a) and 10(b) are a set of graphs illustrating relationships between pulse wave feature amounts 1/VE0.5 of a diabetes patient and a healthy person, which are calculated from each photoplethysmographic signal measured with green light and near-infrared light, and a systolic blood pressure of a wrist in accordance with aspects of the present disclosure;

FIG. 11(a) to 11(d) are a set of graphs illustrating a relationship between each of pulse wave feature amounts ab time and bd time of the diabetes patient and the healthy person, which are calculated from each photoplethysmographic signal measured with green light and near-infrared light, and the systolic blood pressure of the wrist in accordance with aspects of the present disclosure;

FIG. 12(e) to 12(i) are a set of graphs illustrating a relationship between each of pulse wave feature amounts de time and ae time of the diabetes patient and the healthy person, which are calculated from each photoplethysmographic signal measured with the green light and the near-infrared light, and the systolic blood pressure of the wrist, and a relationship between a pulse interval and the systolic blood pressure of the wrist in accordance with aspects of the present disclosure;

FIGS. 13(a) and 13(b) are a set of graphs illustrating a relationship between pulse wave feature amounts (a-b)/(a-d) of the diabetes patient and the healthy person, which are calculated from each photoplethysmographic signal measured with the green light and the near-infrared light and the systolic blood pressure of the wrist in accordance with aspects of the present disclosure;

FIGS. 14(a) and 14(b) are a set of graphs illustrating relationships between pulse wave feature amounts a/S of the diabetes patient and the healthy person, which are calculated from each photoplethysmographic signal measured with green light and near-infrared light, and the systolic blood pressure of the wrist in accordance with aspects of the present disclosure;

FIGS. 15(a) and (b) are a set of graphs illustrating a distribution of a relationship of blood pressure index values calculated for the diabetes patient and the healthy person from a blood pressure index-based expression using the pulse wave feature amounts 1/VE0.5 and de time measured from the green light and the near-infrared light with respect to the systolic blood pressure of the wrist, and a correlation between the blood pressure index value calculated from the blood pressure index-based expression and the systolic blood pressure of the wrist in accordance with aspects of the present disclosure;

FIGS. 16(a) to 16(d) are a set of graphs illustrating a distribution of a relationship of each blood pressure index value with respect to the systolic blood pressure of the wrist, which is calculated for the diabetes patient and the healthy person from the blood pressure index-based expression using the pulse wave feature amounts 1/VE0.5 and de time measured from the green light and the blood pressure index-based expression using the pulse wave feature amounts 1/VE0.5 and de time measured from the near-infrared light, and a correlation between the blood pressure index value calculated from each of the blood pressure index-based expressions and the systolic blood pressure of the wrist in accordance with aspects of the present disclosure;

FIGS. 17(a) and 17(b) are a set of graphs illustrating a relationship between each blood pressure index value calculated from the blood pressure index-based expression using the pulse wave feature amounts 1/VE0.5 and de time measured from the green light and the near-infrared light and the systolic blood pressure of the wrist, when a height of a measurement site from a heart is changed, and a correlation between the blood pressure index value calculated from the blood pressure index-based expression and the systolic blood pressure of the wrist when the height of the measurement site is unified with a height of the heart in accordance with aspects of the present disclosure;

FIGS. 18(a) and 18(b) are a set of graphs illustrating a relationship between the pulse wave feature amount 1/VE0.5 measured from the green light and the systolic blood pressure of the wrist and a relationship between the de time measured from the near-infrared light and the systolic blood pressure of the wrist, when the height of the measurement site from the heart is changed in accordance with aspects of the present disclosure;

FIG. 19 is a graph illustrating a correlation between a blood pressure decrease index value calculated for the diabetes patient and the healthy person from a blood pressure decrease index expression using the pulse wave feature amounts 1/VE0.5 and de time measured from the green light and the near-infrared light and the systolic blood pressure of the wrist in accordance with aspects of the present disclosure;

FIGS. 20(a) and 20(b) are a set of graphs illustrating a distribution of a relationship of a diastolic blood pressure index value calculated for the diabetes patient and the healthy person from a diastolic blood pressure index-based expression using the pulse wave feature amounts 1/VE0.5 and de time measured from the green light and the near-infrared light with respect to a diastolic blood pressure of the wrist, and a correlation between the diastolic blood pressure index value calculated from the diastolic blood pressure index-based expression and the diastolic blood pressure of the wrist in accordance with aspects of the present disclosure;

FIGS. 21(a) and 21(b) are a set of graphs illustrating a distribution of a relationship of a diastolic blood pressure index value calculated for the diabetes patient and the healthy person from a diastolic blood pressure index-based expression using the pulse wave feature amounts 1/VE0.5, de time, and ae time measured from the green light and the near-infrared light with respect to a diastolic blood pressure of the wrist, and a correlation between the diastolic blood pressure index value calculated from the diastolic blood pressure index-based expression and the diastolic blood pressure of the wrist in accordance with aspects of the present disclosure;

FIG. 22 is a flowchart illustrating a flow of processing of a blood pressure estimation method in accordance with aspects of the present disclosure;

FIG. 23 is a diagram illustrating an imaging situation in a method of estimating the height of the measurement site from the heart from an image of a user captured by an imaging device in accordance with aspects of the present disclosure;

FIG. 24 is a diagram illustrating an imaging situation in a method of estimating the height of the measurement site from the heart from the image of the user captured by the imaging device in accordance with aspects of the present disclosure; and

FIG. 25 is a diagram illustrating an example of an image in a method of estimating the height of the measurement site from the heart from the image of the user captured by the imaging device in accordance with aspects of the present disclosure.

### DETAILED DESCRIPTION

Hereinbelow, aspects of the present disclosure will be described. In a following description of the drawings, the same or similar components will be represented with use of the same or similar reference characters. The drawings are exemplary, sizes or shapes of portions are schematic, and technical scope of the present disclosure should not be understood with limitation to the aspects.

Hereinafter, aspects of the present disclosure will be described with reference to the drawings. Here, the same reference signs denote the same constituent elements, and redundant description thereof will be omitted.

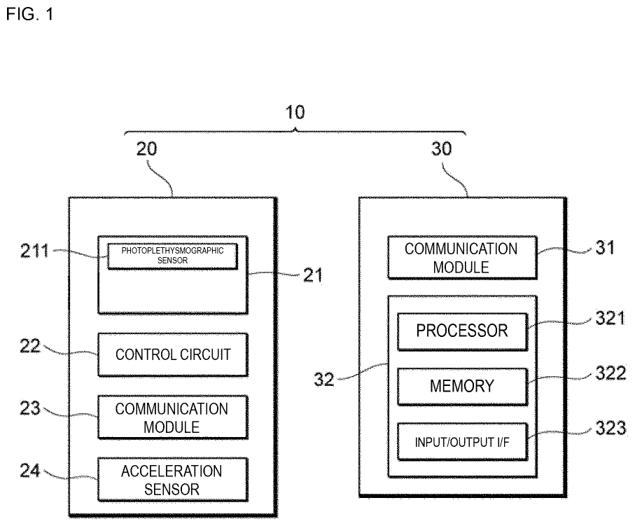

FIG. 1 is an explanatory diagram illustrating a configuration of a biological information measurement system 10 according to an aspect of the present disclosure. The biological information measurement system 10 includes a sensing device 20 that measures biological information of a user who is a subject and a computer 30 that is configured to be communicable with the sensing device 20.

The sensing device 20 is, for example, a wearable device having a structure that allows it to be mounted on a peripheral site (for example, a finger) of a user. The sensing device 20 includes a biological sensor 21 that measures biological information from a peripheral site (for example, a finger) of the user, a control circuit 22 that controls operation of the biological sensor 21, a communication module 23 that transmits a measurement result of the sensing device 20 to the computer 30 via a wireless network or a wired network, and an acceleration sensor 24 that measures a movement acceleration of the sensing device 20.

The biological sensor 21 includes, for example, a photoplethysmographic sensor 211 that measures an index value indicating a peripheral blood pressure of a user. The peripheral blood pressure in the present disclosure denotes the blood pressure of a capillary or an arteriole of a periphery. In addition, in the present disclosure, an index indicating a blood pressure in an arteriole and a capillary, particularly in the capillary, is referred to as a peripheral blood pressure index. Here, the arteriole is, for example, a thin artery having a diameter of about 20 to 200 μm, and is a blood vessel existing between the artery and the capillary. In addition, the capillary is, for example, a thin blood vessel having a diameter of about 10 μm, and is a blood vessel connecting an artery and a vein.

The term “peripheral blood pressure” may also be used to mean the blood pressure of a wrist and the blood pressure of an ankle, which are measured by a cuff-type sphygmomanometer. In this case, the peripheral blood pressure is a measurement value at a thick artery (radial artery or the like) and is different from the blood pressure in the arteriole and the capillary in the present disclosure. The blood pressure in the thick artery is generally the blood pressure measured by a cuff-type sphygmomanometer, and the blood pressure in a blood vessel is reduced as the blood moves from the artery to the arteriole and the capillary. The degree of the blood pressure decrease varies depending on a measurement site, a blood vessel state of the individual (artery solidification or the like), a mental state (autonomic nerve state or the like), an environment (temperature, noise, or the like), clothing, and the like.

The following two points (1) and (2) are assumed as features of the peripheral blood pressure index. (1) in a case where the blood vessel is healthy, the peripheral blood pressure index is substantially proportional to the blood pressure (in the upper arm or the wrist) under a condition in which the blood vessel resistance does not change. (2) In a case where the blood vessel is contracted by cooling the vicinity of the measurement site, the peripheral blood pressure index is reduced. This means that the blood vessel resistance of the periphery increases. Thus, the blood pressure in the upper arm or the wrist may be increased.

The photoplethysmographic sensor 211 is equipped with three LEDs as light sources and measures a photoplethysmographic signal at three wavelengths (green, red, and near-infrared). Oxidized hemoglobin exists in the blood of the artery, and the blood of the artery has the property of absorbing incident light. Thus, a photoplethysmographic signal can be measured by sensing a blood flow rate (change in volume of the blood vessel) that changes with the pulsation of the heart in a time-series manner. The red LED is mounted for calculating the oxygen saturation, and may not be essential for extracting the peripheral blood pressure index. The photoplethysmographic sensor 211 is equipped with a photodiode (PD) as a light receiving element, sequentially emits light from the three LEDs in a time-division manner to irradiate the skin of the finger, and receives light that has been reflected and scattered by the PD.

The communication module 23 transmits a measurement result of the sensing device 20 (for example, a photoplethysmographic signal measured by the photoplethysmographic sensor 211, an acceleration of the sensing device 20 measured by the acceleration sensor 24, and the like) to the computer 30 via a wireless network or a wired network.

The acceleration sensor 24 measures the movement acceleration of the sensing device 20 when the user changes the posture in order to measure the pulse wave signal. The acceleration sensor 24 is a three-axis acceleration sensor that detects a direction in which a gravitational acceleration is applied. A detection signal thereof is used for estimating a height at which the user attaches the sensing device 20, estimating a position at which the user attaches the sensing device 20 (for example, a position of the heart of the user), or estimating a posture of the user, such as a standing posture (orthostatic position), a sitting posture (sitting position), or a posture lying on the back (supine position).

The computer 30 is, for example, a multifunctional mobile phone called a smartphone or a general-purpose computer (for example, a notebook personal computer, a desktop personal computer, a tablet terminal, or a server computer). The computer 30 includes a communication module 31 that receives the measurement result of the biological sensor 21 from the sensing device 20 via a wireless network or a wired network, and a signal processing device 32 that performs processing of estimating the biological information of the user from the measurement result of the biological sensor 21. The signal processing device 32 includes a processor 321, a memory 322, and an input/output interface 323.

The signal processing device 32 performs first-order differentiation (velocity pulse wave) and second-order differentiation (acceleration pulse wave) on two photoplethysmographic waves (volume pulse waves) measured by the green LED and the near-infrared LED and calculates a pulse wave feature amount by dividing each of the photoplethysmographic waves into each beat. Then, the peripheral blood pressure index is calculated based on the pulse wave feature amount. In addition, the signal processing device 32 estimates the height of the site of the user to which the sensing device 20 is attached or estimates the posture of the user based on the signal from the acceleration sensor 24.

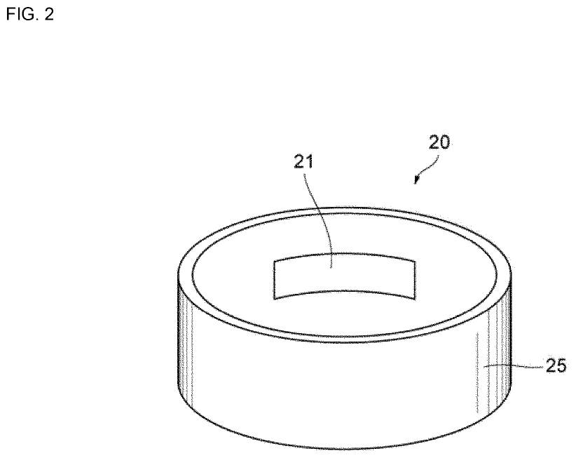

FIG. 2 is an explanatory diagram illustrating an external configuration of the sensing device 20 according to the aspects of the present disclosure. The measurement site of a photoplethysmographic wave may be a wrist, a neck, a face, an ear, or the like, and a finger is recommended. One reason why the finger is recommended is that the finger has a relatively thin epidermis and therefore it is easy to measure the photoplethysmographic wave, and another reason is that the value of each feature amount is likely to be stable because the route of the capillary is less complicated than that in the face or the like. As a device for measuring the photoplethysmographic wave, a ring-type wearable device that includes an optical sensor and is mounted on the finger is recommended. This is because, in a case of continuous or intermittent measurement, the discomfort or unpleasant feeling is small even in a case of being mounted for a long time. The measurement site is not limited to the finger, and the wearable device may be a wristband type to be mounted on the wrist, a wristwatch type, an earphone type to be mounted on the ear, a patch type to be stuck to the skin, or a neckband type to be mounted on the neck. In addition, the device may not necessarily be a wearable device and may be a portable device such as a smartphone or a stationary device having a configuration in which the measurement is performed by placing the finger on the sensor.

In the present aspect, the sensing device 20 includes a ring-shaped housing 25 configured to be mounted on the finger of the user. For example, the housing 25 has a hollow cylindrical shape in the example illustrated in FIG. 2. The biological sensor 21 is attached to an inner peripheral surface (an inner surface of the hollow cylinder) of the housing 25 such that a ball of the figure of the user faces the biological sensor 21 in a case where the sensing device 20 is mounted on the finger of the user. The shape of the housing 25 is not limited to the hollow cylindrical shape, and for example, may be a tubular shape that fits on the finger of the user (for example, a finger sack shape), and a bottom (portion where the fingertip comes into contact with) of a tube may be present or absent.

FIG. 3 is an example of a posture of a user 40 when biological information is measured. In this example, the user 40 is in a state in which the finger on which the sensing device 20 is mounted is stationary at the position of the heart 41, and the sensing device 20 measures the biological information from the finger of the user 40. The position (measurement position) of the sensing device 20 when the biological information is measured is not limited to the position of the chest (heart) 41 of the user 40, and may be a position of the face (forehead) or a position of the abdomen (navel) of the user 40. In addition, the posture of the user 40 when the biological information is measured may be a posture of a sitting position or a posture of a supine position.

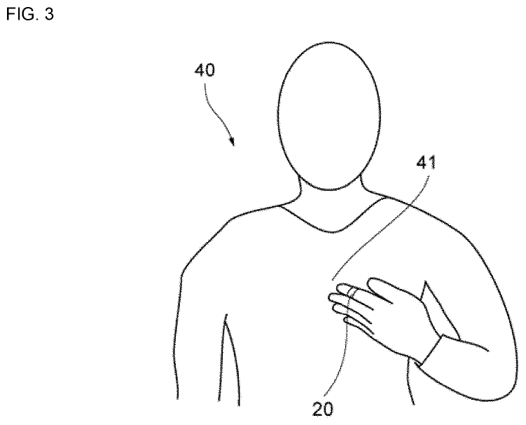

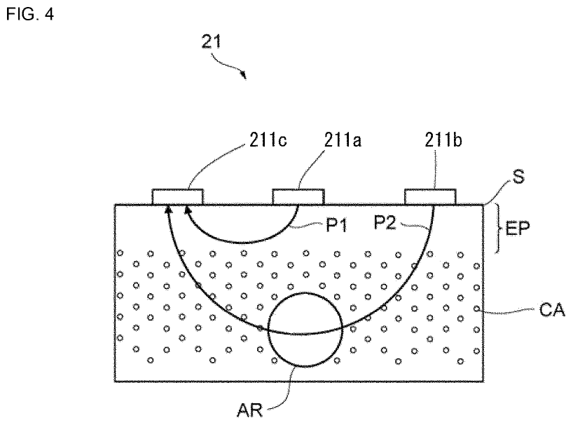

The acquisition of the photoplethysmographic signal by the biological sensor 21 will be described with reference to FIG. 4. FIG. 4 is a schematic cross-sectional view of a state in which the biological sensor 21 is attached close to a body surface S of the user.

The biological sensor 21 includes light emitting elements 211a and 211b and a light receiving element 211c. The biological sensor 21 irradiates the body surface S with light and receives light absorbed or reflected by an epidermis region EP of the user, a plurality of capillaries CA, and an arteriole AR from which each capillary CA branches off. In the present aspect, a case where one light receiving element 211c is provided for the light emitting element 211a that is a first light source and the light emitting element 211b that is a second light source will be described. A light receiving element may be provided for each of the light emitting elements 211a and 211b.

As the light emitting element 211a that is the first light source, for example, an LED or a laser having a wavelength in the vicinity of blue to yellow-green (preferably a wavelength in the vicinity of 500 to 550 nm) is desirable. In the present aspect, the light emitting element 211a is a green LED. As the light emitting element 211b that is the second light source, for example, an LED or a laser having a wavelength in the vicinity of red to near-infrared (preferably a wavelength in the vicinity of 750 to 950 nm) is desirable. In the present aspect, the light emitting element 211b is a near-infrared LED. The light emitting element 211a emits light in a wavelength range that is strongly absorbed in the living body, and the light emitting element 211b emits light in a wavelength range that is relatively weakly absorbed in the living body. Description will be made on the assumption that the light emitting element 211a is a green LED 211a, and the light emitting element 211b is a near-infrared LED 211b. A photodiode (PD) or a phototransistor is used as the light receiving element 211c. A Si photodiode is recommended.

The green LED 211a is provided at a position closer to the light receiving element 211c than is the near-infrared LED 211b. For example, it is recommended that the distance between the green LED 211a and the light receiving element 211c be about 1 to 3 mm, and the distance between the near-infrared LED 211b and the light receiving element 211c be about 5 to 20 mm. By providing the green LED 211a at the position closer to the light receiving element 211c than is the near-infrared LED 211b, a light reception signal based on the light from the green LED 211a can contain more information of a shallow region of the skin than a light reception signal based on the light from the near-infrared LED 211b.

The light emitted from the green LED 211a is absorbed by the epidermis region EP of the user and the capillary CA on the epidermis region EP side, and the transmitted light or the reflected light is detected by the light receiving element 211c. The light emitted from the near-infrared LED 211b is absorbed by the epidermis region EP of the user, the capillary CA, and the arteriole AR located on the inside of the body with respect to the epidermis region EP, and is detected by the light receiving element 211c. In FIG. 4, the light from the green LED 211a is schematically illustrated as light along an optical path P1, and the light from the near-infrared LED 211b is schematically illustrated as light along an optical path P2.

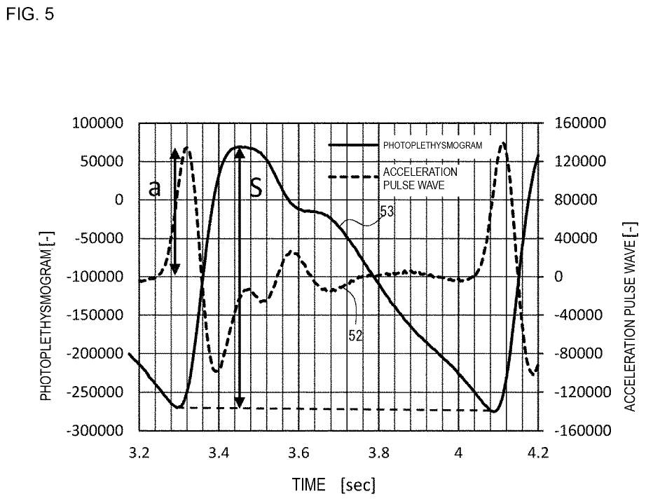

The graph of FIG. 5 illustrates an acceleration pulse wave signal 52 obtained by performing second-order differentiation on the photoplethysmographic signal 53. In the graph, the horizontal axis represents time [sec], and the vertical axis represents the signal intensity of the acceleration pulse wave signal 52 and the photoplethysmographic signal 53. As illustrated in the figure, in the photoplethysmographic signal 53 has a pulse wave height (maximum amplitude value) S that is a height of a maximal point after minimal points are joined by a straight line and inclination correction is performed such as the inclination of the straight line becomes 0.

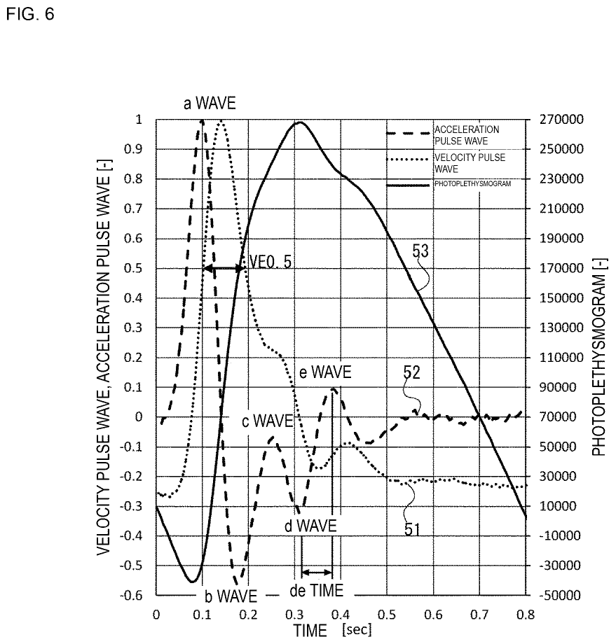

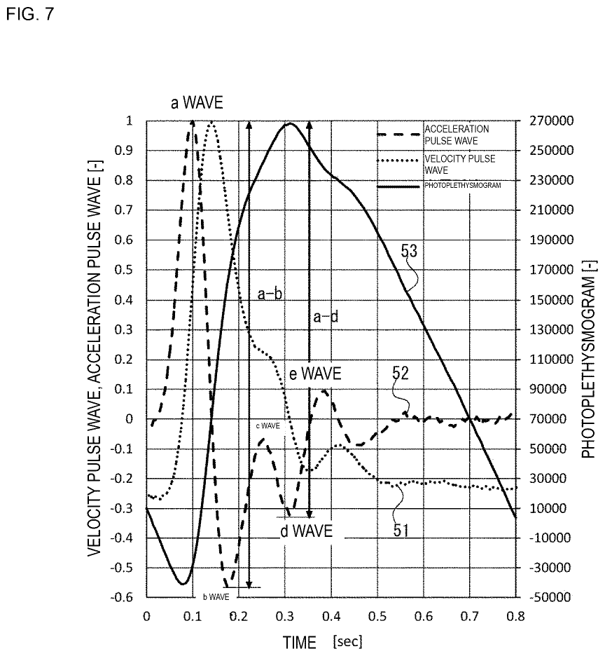

In addition, as illustrated in the graph of FIG. 6, a waveform width at a half value of a maximum peak value of the velocity pulse wave signal 51 obtained by performing first-order differentiation on the photoplethysmographic signal 53 is referred to as VE0.5. In the graph, the horizontal axis represents time [sec], and the vertical axis represents the signal intensity of the velocity pulse wave signal 51, the acceleration pulse wave signal 52, and the photoplethysmographic signal 53. Normalization processing in which the maximum value of each of the velocity pulse wave signal 51 and the acceleration pulse wave signal 52 is set to 1 is performed. The peaks (maximal and minimal peaks) of the acceleration pulse wave signal 52 are referred to as an a wave, a b wave, a c wave, a d wave, and an e wave, as illustrated in the figure. The waveform is such that the a wave, the c wave, and the e wave have peaks protruding on the positive side and the b wave and the d wave have peaks protruding on the negative side. In addition, a difference between a d-wave peak time and an e-wave peak time is referred to as a de time. In addition, the signal intensities of peak vertices of the a wave, the b wave, the c wave, the d wave, and the e wave are a, b, c, d, and e, respectively. In addition, as illustrated in the graph of FIG. 7, a peak difference between the a wave and the b wave of the acceleration pulse wave signal 52 is a-b, and a peak difference between the a wave and the d wave is a-d. The horizontal axis and the vertical axis of the graph are the same as those in FIG. 6.

As pulse wave feature amounts illustrating the feature (1) above that the peripheral blood pressure index is substantially proportional to the blood pressure of the upper arm or the wrist, the following three items are extracted:
1/VE0.5
a/S
(a-b)/(a-d)

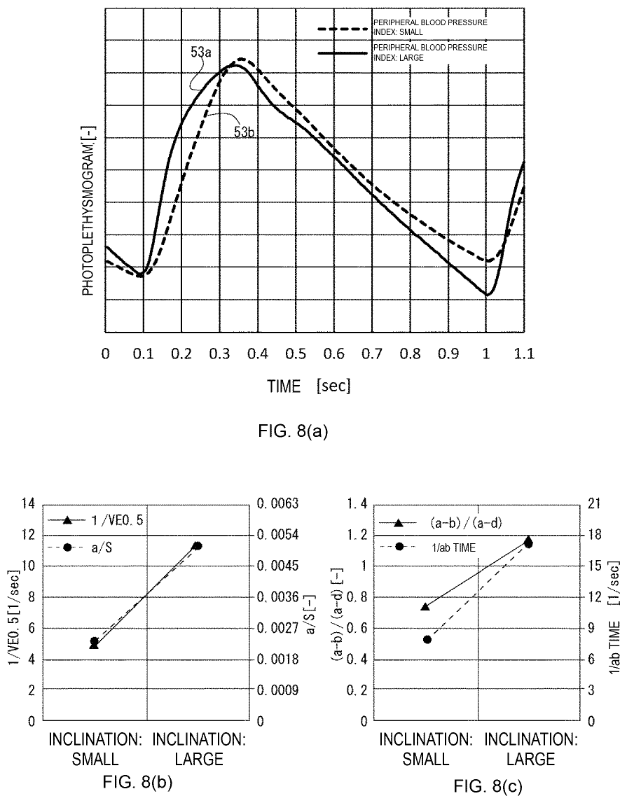

As illustrated in FIG. 8, these pulse wave feature amounts are related to the steepness of rising of the waveform of the photoplethysmographic signal 53. FIG. 8(a) is a graph illustrating two photoplethysmographic signals 53a and 53b having different steepnesses of the rising of a photoplethysmographic waveform. In the graph, the horizontal axis represents time [sec], and the vertical axis represents the signal intensity of the photoplethysmographic signal 53. It is understood that, of the two photoplethysmographic signals 53a and 53b, the photoplethysmographic signal 53a indicated by the solid line has a steeper rising (inclination: large) than the photoplethysmographic signal 53b indicated by the broken line.

The graph illustrated in FIG. 8(b) shows the change in each value of the pulse wave feature amount 1/VE0.5 and the pulse wave feature amount a/S due to the difference in the inclination of the photoplethysmographic signals 53a and 53b. The vertical axis of the graph represents each value of the pulse wave feature amount 1/VE0.5 and the pulse wave feature amount a/S, and the horizontal axis is divided into the photoplethysmographic signal 53b having a small inclination and the photoplethysmographic signal 53a having a large inclination. From the graph, it is understood that each of the pulse wave feature amounts for the photoplethysmographic signal 53a having a large inclination is larger than each of the pulse wave feature amounts for the photoplethysmographic signal 53b having a small inclination, for both the pulse wave feature amount 1/VE0.5 and the pulse wave feature amount a/S.

The graph illustrated in FIG. 8(c) shows the change in each value of the pulse wave feature amount (a-b)/(a-d) and the pulse wave feature amount 1/ab time due to the difference in the inclination of the photoplethysmographic signals 53a and 53b. The vertical axis of the graph represents each value of the pulse wave feature amount (a-b)/(a-d) and the pulse wave feature amount 1/ab time, and the horizontal axis is divided into the photoplethysmographic signal 53b having a small inclination and the photoplethysmographic signal 53a having a large inclination. From the graph, it is understood that both the pulse wave feature amount (a-b)/(a-d) and the pulse wave feature amount 1/ab time for the photoplethysmographic signal 53a having a large inclination are larger than the respective pulse wave feature amounts for the photoplethysmographic signal 53b having a small inclination.

Therefore, it can be confirmed that the three pulse wave feature amounts 1/VE0.5, a/S, and (a-b)/(a-d) given above are related to the steepness of the rising of the photoplethysmographic waveform. That is, the steepness of the rising of the photoplethysmographic waveform can be represented by the pulse wave feature amounts, and the pulse wave feature amounts are assumed to be the pulse wave feature amounts illustrating the feature (1) described above. The pulse wave feature amount 1/ab time is added for comparison as another feature amount related to the steepness of the rising of the photoplethysmographic waveform.

The pulse wave feature amounts 1/VE0.5, a/S, and (a-b)/(a-d) that are the basis of the peripheral blood pressure index may be used alone. The values of the respective peak values a, b, c, and d of the a wave, the b wave, the c wave, and the d wave are easily affected by a pressing state of the photoplethysmographic sensor 211 on the skin or a body movement noise, and there is a large variation due to individual differences. Therefore, since 1/VE0.5 is a feature amount that can be relatively stably obtained among the above pulse wave feature amounts, it is desirable to use 1/VE0.5 alone or to use 1/VE0.5 as a base and use other feature amounts in a supplementary manner. Furthermore, a value obtained by weighting each of the pulse wave feature amounts and performing averaging processing may be used, or a value obtained by normalizing the magnitude of each pulse wave feature amount and performing averaging processing may be used.

In the present aspect, an attempt is made to derive a blood pressure estimation expression configured for estimating the blood pressure value of the user by performing a calculation. In order to create the blood pressure estimation expression, the following data is collected in order to extract the pulse wave feature amount having a high possibility of having a causal relationship with the blood pressure. Unless otherwise specified, the blood pressure is a wrist systolic blood pressure below. (A) An experiment of intentionally changing the blood pressure by changing the height of a blood pressure measurement site from the heart was performed, and correlation data between each of the above-described pulse wave feature amounts and the blood pressure was collected by changing the height of the blood pressure measurement site from the heart. In addition, correlation data between each of the above-described pulse wave feature amounts and the blood pressure was collected by forcibly contracting the blood vessel by cooling the vicinity of the blood pressure measurement site. (B) In addition, with the cooperation of a hospital, correlation data between each pulse wave feature amount and the blood pressure was acquired for a diabetes patient and a healthy person. Thus, extreme correlation data varying greatly depending on different users was collected.

First, as the experiment of intentionally changing the blood pressure in (A), the following experiment was performed on the healthy person as the target.

That is, the finger-mounting type sensing device 20 illustrated in FIG. 2 equipped with the photoplethysmographic sensor 211 was prepared, a wrist-type cuff sphygmomanometer was mounted on the left wrist (may also be mounted on the right wrist) of the user 40, and the sensing device 20 was attached to the index finger (may also be attached to another finger) of the left hand. Then, in a resting sitting position, the left hand on which the sensing device 20 was mounted was held at the height of the abdomen (navel), the height of the chest, and the height of the face (forehead), and the photoplethysmographic wave and the blood pressure were measured. The measurement site of the photoplethysmographic wave is the ventral side of a fingertip (distal phalanx). When the photoplethysmographic wave and the blood pressure were measured simultaneously, the blood flow of the finger was inhibited by the cuff, so that the blood pressure was measured after the measurement of the photoplethysmographic wave was ended. Then, the left elbow was cooled with a cooling agent in a state in which the left hand was held at the chest height. After cooling for several minutes, the photoplethysmographic wave and the blood pressure was measured. From the photoplethysmographic wave measured in this manner, the feature amount of the pulse wave illustrating the features of the peripheral blood pressure index of (1) and (2) described above was calculated as follows.

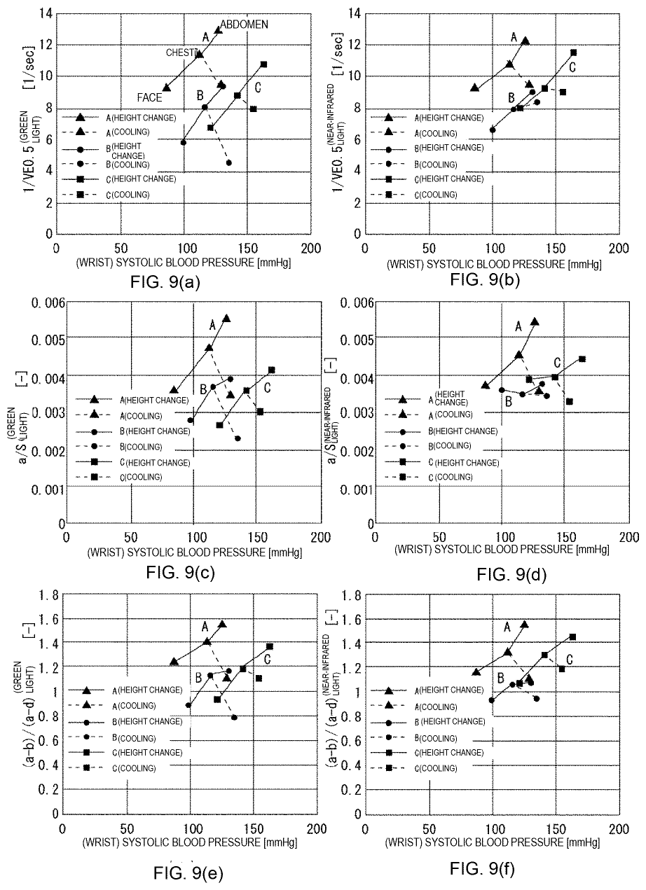

FIG. 9 shows a relationship between the systolic blood pressure and each pulse wave feature amount when the height of the measurement site (finger) from the heart is changed, and a relationship between the systolic blood pressure and each pulse wave feature amount when the vicinity of the elbow of the arm on the side of the finger, which is the measurement site, is cooled at the height of the chest, which are measured by the measurement method described above. In addition, FIGS. 9(a), 9(c), and 9(e) show the results calculated from the photoplethysmographic signal measured with the green light emitted from the green LED 211a for the pulse wave feature amounts 1/VE0.5, a/S, and (a-b)/(a-d), respectively. In addition, FIGS. 9(b), 9(d), and 9(f) show the results calculated from the photoplethysmographic signal measured with the near-infrared light emitted from the near-infrared LED 211b for the pulse wave feature amounts 1/VE0.5, a/S, and (a-b)/(a-d), respectively.

The horizontal axis of each graph is the systolic blood pressure [mmHg] measured at the wrist, and the vertical axis is the magnitude of each pulse wave feature amount. In addition, the measurement is performed for three users A, B, and C. A characteristic line A obtained by linking triangular plots shows the measurement result when the height of the measurement site (finger) from the heart is changed for the user A. A characteristic line B obtained by linking circular plots shows the measurement result when the height of the measurement site (finger) from the heart is changed for the user B. A characteristic line C obtained by linking square plots shows the measurement result when the height of the measurement site (finger) from the heart is changed for the user C. In addition, each plot indicated by the broken line drawn out shows the measurement result when the vicinity of the measurement site is cooled at the chest height.

It is understood from each of the characteristic lines A, B, and C that each of the pulse wave feature amounts illustrated in FIGS. 9(a), 9(c), and 9(e), which are calculated from the photoplethysmographic signal measured with the green light, show a tendency that the systolic blood pressure and each of the pulse wave feature amounts are close to being proportional to each other, when the height of the measurement site (finger) from the heart is changed. As the height of the measurement site (finger) from the heart becomes higher from the abdomen to the chest and then to the face, the systolic blood pressure is reduced substantially in proportion to the reduction in each of the pulse wave feature amounts. In addition, when the vicinity of the measurement site is cooled, the magnitude of each pulse wave feature amount is reduced, and a tendency that the systolic blood pressure increases can be confirmed from each plot illustrated by the broken line. This matches the above-described features (1) and (2) of the peripheral blood pressure index assumed.

On the other hand, it is understood that the calculation results of the respective pulse wave feature amounts illustrated in FIGS. 9(b), 9(d), and 9(f), which are calculated from the photoplethysmographic signal measured with the green light and the near-infrared light substantially simultaneously, are not clear in the above-described tendency as compared with the results calculated from the green light. As a result, it is estimated that the use of the photoplethysmographic signal acquired with the green light is more suitable for the peripheral blood pressure index as compared with the near-infrared light. From this experiment, the peripheral blood pressure index was created from the pulse wave feature amounts 1/VE0.5, a/S, and (a-b)/(a-d).

In addition, the following experiment was performed in collaboration with a hospital by targeting a diabetes patient, as the experiment of collecting the different large correlation data in (B).

That is, the wrist-type cuff sphygmomanometer was mounted on the left wrist (or the right wrist) of the user 40 who is a diabetes patient, and the finger-mounting type sensing device 20 illustrated in FIG. 2 was mounted on the index finger (or other fingers) of the left hand. Then, in a resting sitting position, the left hand on which the sensing device 20 was mounted was held at the height of the abdomen (navel), the height of the chest, and the height of the face (forehead), and the photoplethysmographic wave and the blood pressure were measured. The measurement site of the photoplethysmographic wave is the ventral side of a fingertip (distal phalanx). When the photoplethysmographic wave and the blood pressure were measured simultaneously, the blood flow of the finger was inhibited by the cuff, so that the blood pressure was measured after the measurement of the photoplethysmographic wave was ended.

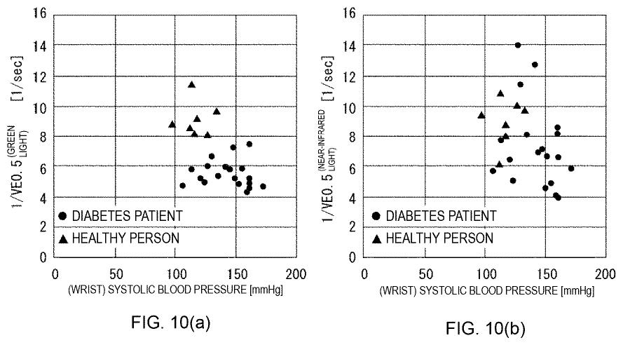

From the photoplethysmographic waves measured in this manner, the relationship between the pulse wave feature amount 1/VE0.5 and the systolic blood pressure was calculated as illustrated in the graphs of FIG. 10(a) and FIG. 10(b). The horizontal axis of each graph is the systolic blood pressure of the wrist, and the vertical axis is the magnitude of the pulse wave feature amount 1/VE0.5. The graph of FIG. 10(a) shows the pulse wave feature amount 1/VE0.5 calculated from the photoplethysmographic signal measured using the green light, and the graph of FIG. 10(b) shows the pulse wave feature amount 1/VE0.5 calculated from the photoplethysmographic signal measured using the near-infrared light. For comparison, data of the healthy person described above is also plotted on the graph. Data of the diabetes patient is illustrated by a circular plot, and the data of the healthy person is illustrated by a triangular plot.

In the graphs of FIG. 10, the pulse wave feature amount 1/VE0.5 tends to be smaller as the blood pressure is higher. In FIG. 10(a), it is understood that the diabetes patient is concentrated at the low value of the pulse wave feature amount 1/VE0.5, and the appearance of the pulse wave feature amount 1/VE0.5 is clearly isolated between the healthy person and the diabetes patient. It is estimated that this is due to the following mechanism.

That is, when a state in which the blood glucose level is high continues, a so-called vascular disease in which blood vessels become brittle and crumble occurs. In this vascular disease, artery solidification progresses in thick blood vessels, and the function of the blood vessels (vascular endothelial function) is deteriorated due to damage to thin blood vessels, resulting in poor blood flow. The local blood pressure (peripheral blood pressure) is reduced as the blood progresses from the thick artery to the arteriole and the capillary, and it is presumed that the degree of reduction in the peripheral blood pressure increases when the vascular function (vascular endothelial function) is reduced. It is said that 40% to 60% of diabetes patients have complications of hypertension. In FIG. 10(a), the diabetes patient has a higher relative systolic blood pressure than the healthy person, but the tendency may not be remarkable. However, the tendency that the pulse wave feature amount 1/VE0.5 (peripheral blood pressure index) is low in the diabetes patient is clear. This can be explained by the fact that the peripheral (capillary) blood pressure is reduced because the peripheral blood vessel disorder occurs in the diabetes patient and the blood does not easily flow to the periphery (capillary).

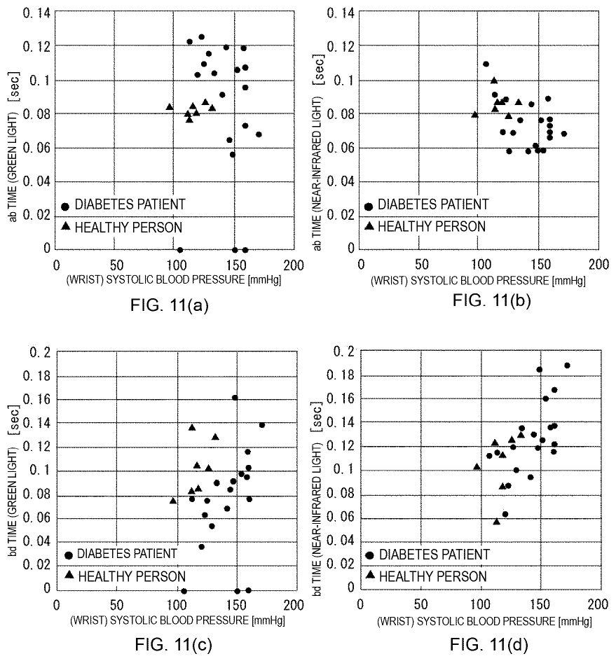

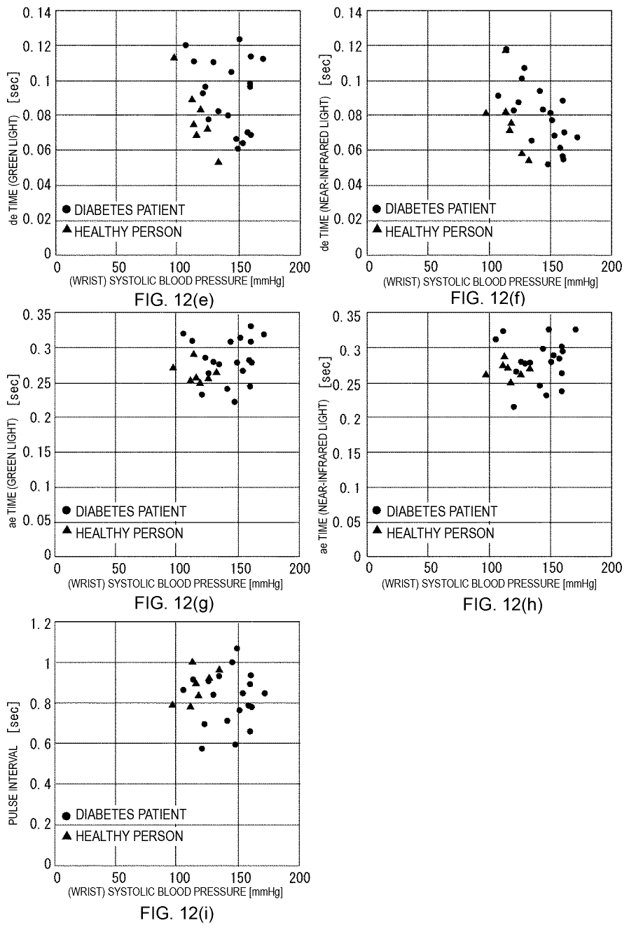

FIG. 11(a) to 11(d) and FIG. 12(e) to 12(h) are graphs illustrating the relationship between each pulse wave feature amount and the systolic blood pressure, which are obtained by the above-described experiment performed on the diabetes patient as the target. FIG. 12(i) is a graph illustrating the relationship between a pulse interval and the systolic blood pressure. For comparison, the data of the healthy person described above is also plotted on each of these graphs. The data of the diabetes patient is illustrated by a circular plot, and the data of the healthy person is illustrated by a triangular plot.

In FIG. 11(a) to 11(d) and FIG. 12(e) to 12(h), the horizontal axis of each graph is the systolic blood pressure of the wrist, and the vertical axis is the magnitude of each pulse wave feature amount. In FIG. 12(i), the horizontal axis of the graph is the systolic blood pressure of the wrist, and the vertical axis is the pulse interval. In each of the graphs of FIGS. 11(a) and 11(b), the pulse wave feature amount is a pulse wave feature amount ab time calculated from the photoplethysmographic signal measured using the green light and the near-infrared light. In each of the graphs of FIGS. 11(c) and 11(d), the pulse wave feature amount is a pulse wave feature amount bd time calculated from the photoplethysmographic signal measured using the green light and the near-infrared light. In each of the graphs of FIGS. 12(e) and 12(f), the pulse wave feature amount is a pulse wave feature amount de time calculated from the photoplethysmographic signal measured using the green light and the near-infrared light. Each of the graphs of FIGS. 12(g) and 12(h) is a pulse wave feature amount ae time calculated from the photoplethysmographic signal measured using the green light and the near-infrared light.

Here, the ab time is a difference between the a-wave peak time and the b-wave peak time of the acceleration pulse wave signal 52 illustrated in FIG. 6, the bd time is a difference between the b-wave peak time and the d-wave peak time, and the ae time is a difference between the a-wave peak time and the e-wave peak time.

In each of the graphs of FIG. 11(a) to 11(d) and FIG. 12(e) to 12(h), the pulse wave feature amount illustrating the correlation with the systolic blood pressure is the de time illustrating the negative correlation illustrated in FIGS. 12(e) and 12(f), the bd time illustrating the positive correlation illustrated in FIGS. 11(c) and 11(d), and the ab time illustrating the weak negative correlation illustrated in FIGS. 11(a) and 11(b). The ae time illustrated in FIGS. 12(g) and 12(h) and the pulse interval illustrated in FIG. 12(i) does not show the correlation with the systolic blood pressure. In addition, the pulse wave feature amount illustrating a difference between the healthy person and the diabetes patient is the de time which shows a tendency that the pulse wave feature amount for the diabetes patient is larger, and a clear tendency may not be confirmed in the other feature amounts.

The mechanism of the de time changing is estimated as follows. From FIG. 7, it is understood that the d-wave peak time is close to the time of the maximal value of the photoplethysmographic signal 53. A position where the photoplethysmographic signal 53 has the maximal value may be in the vicinity of the b wave. The waveform in the vicinity of the b wave is considered to be an ejection wave from the heart, and the waveform in the vicinity of the d wave is considered to be the reflected wave from the periphery. A recessed portion of the photoplethysmographic signal 53 after the photoplethysmographic signal 53 has the maximal value, near 0.4 sec in FIG. 7, is referred to as a notch. The tendency that the de time is shortened as the blood pressure increases means that the d-wave position is moved to the rear (e-wave peak direction) because no clear tendency is illustrated between the blood pressure and the ae time.

The increase in the blood flow rate means an increase in the ejection wave and the reflected wave. Therefore, it is presumed that the protruding portion (in the vicinity of the b wave to the d wave) of the photoplethysmographic signal 53 is spread to the rear, and as a result, the d-wave position is moved to the rear. That is, it is presumed that the blood flow rate increases because the blood pressure has increased, and the de time becomes shorter due to the increase in the blood flow rate. In addition, from FIG. 12(f), the de time tends to be larger in the diabetes patient than in the healthy person. Therefore, the d-wave position tends to approach the e wave as the blood pressure increases, and the d-wave position tends to be farther from the e wave in the diabetes patient than in the healthy person, that is, the blood flow rate tends to be lower in the diabetes patient than in the healthy person.

From the above presumption, it can be presumed that the blood flow rate is low in the diabetes patient. This does not contradict the above-described presumption that the blood pressure of the periphery is reduced and the blood is less likely to flow in the diabetes patient.

From the above description, the pulse wave feature amount in which the clear differences between the diabetes patient and the healthy person are confirmed is the pulse wave feature amounts 1/VE0.5 and de time obtained by measuring the photoplethysmographic wave with the green light.

In FIGS. 11(a) and 11(c), a plurality of circular plots are illustrated on the time axis of the horizontal axis, which shows that the b wave could not be detected. All of the plots are plots of diabetes patients. As described above, in a person with poor peripheral blood circulation, such as a diabetes patient, the b wave is small, and it is often difficult to detect the b wave.

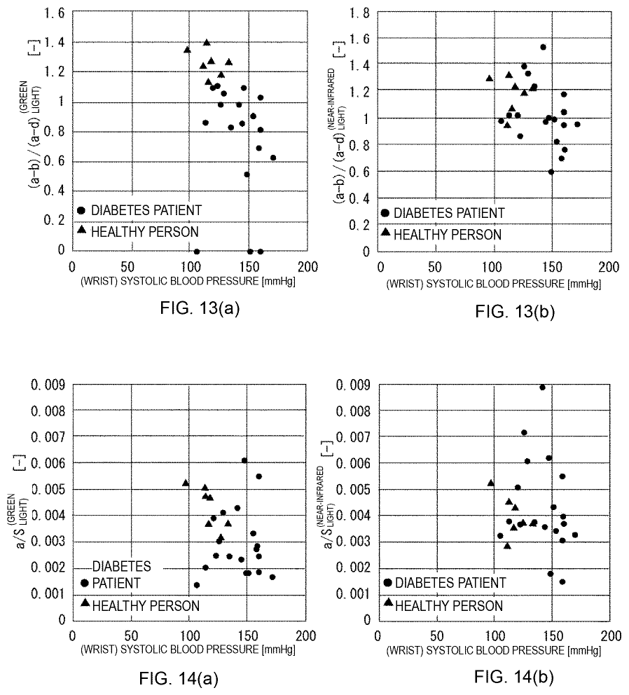

The graphs of FIG. 13 show the correlation relationship between the pulse wave feature amount (a-b)/(a-d) and the (wrist) systolic blood pressure. In the graphs, the horizontal axis represents the systolic blood pressure of the wrist, and the vertical axis represents the magnitude of the pulse wave feature amount (a-b)/(a-d). In addition, the graph of FIG. 13(a) shows a correlation relationship between the blood pressure and the pulse wave feature amount (a-b)/(a-d) calculated from the photoplethysmographic signal measured using green light, and the graph of FIG. 13(b) shows a correlation relationship between the blood pressure and the pulse wave feature amount (a-b)/(a-d) calculated from the photoplethysmographic signal measured using the near-infrared light.

The tendency that the magnitude in the pulse wave feature amount (a-b)/(a-d) is reduced as the blood pressure increases is the same as the pulse wave feature amount 1/VE0.5. In the graph of FIG. 13(a) obtained from the measurement using the green light, the diabetes patient and the healthy person are clearly isolated. In this graph as well, a plurality of circular plots are seen on the time axis of the horizontal axis, which shows that the b wave could not be detected.

In addition, the graphs of FIG. 14 show a relationship between the pulse wave feature amount a/S and the (wrist) systolic blood pressure. In the graphs, the horizontal axis is the systolic blood pressure of the wrist, and the vertical axis is the magnitude of the pulse wave feature amount a/S. In addition, the graph of FIG. 14(a) shows a correlation relationship between the blood pressure and the pulse wave feature amount a/S calculated from the photoplethysmographic signal measured using green light, and the graph of FIG. 14(b) shows a correlation relationship between the blood pressure and the pulse wave feature amount a/S calculated from the photoplethysmographic signal measured using the near-infrared light. The above-described tendency that the magnitude of the pulse wave feature amount a/S is reduced as the blood pressure increases is not clear. This is because there is a large variation in the calculation results of the pulse wave feature amount in the diabetes patient. It is estimated that the cause of the variation is that each value of a and S is easily affected by various factors.

From the above data collection results, the pulse wave feature amount illustrating the difference between the diabetes patient and the healthy person is 1/VE0.5 and (a-b)/(a-d) which are the peripheral blood pressure index, and the de time. It is estimated that these pulse wave feature amounts are feature amounts with a high possibility of having a causal relationship with the blood pressure.

Next, based on these pulse wave feature amounts, first, a blood pressure index-based expression is created. Finally, it is planned to improve the estimation accuracy by performing parameter adjustment using a large number of pieces of data based on the base expression. A blood pressure index-based expression proposal will be created with centering on the pulse wave feature amounts 1/VE0.5 and de time.

First, the basic specifications of the blood pressure index-based expression are set as follows: (a) The blood pressure value at the height of the chest (heart) as the measurement site is estimated. This is because, even if the wrist blood pressure value when the measurement site is other than the chest height can be estimated, the wrist blood pressure value may not be valuable for the user. (b) The measurement site is limited to the fingertip. It is assumed that the ring device is mounted in consideration of usability.

Thus, the ring device mounted on the finger is designed to be able to estimate the blood pressure value when the ring device is held at the height of the chest (heart). If the measurement is made when the ring device is shifted from the height of the heart, it is considered that the desirable specification of the blood pressure index-based expression is that the blood pressure value at the height of the heart can always be estimated, and that the estimated blood pressure value is not decreased (increased) according to the hydrostatic head difference when the ring device is at a position increased (decreased) from the height of the heart. However, since it is difficult to estimate the blood pressure value at the height of the heart regardless of the height of the ring device from the heart, the estimation accuracy in a case where the ring device is shifted from the height of the heart is not required, that is, is not guaranteed. The blood pressure is 7 to 8 mmHg lower if the measurement site is 10 cm higher than the height of the heart. That is, if the height range to be regarded as being equal to the height of the heart is ±10 cm, the blood pressure value varies by ±7 to 8 mmHg only with that.

The blood pressure index-based expression created with the above-described idea is described in Expression (1) as follows.

$${( 1 / VE 0.5 )}_{a}^{- α} \times de {TIME}_{b}^{- β} \qquad ( 1 )$$

Here, the subscripts a and b represent the meaning of the green light or the near-infrared light, and represent the emission color of a measurement light source of the photoplethysmographic signal used for the calculation of the pulse wave feature amount 1/VE0.5 or de time. In addition, the exponents α and β indicating the power are positive numerical values. In a case where the calculation result of Expression (1) is used as the blood pressure estimation value, a proportional coefficient is further multiplied by the blood pressure index value calculated by Expression (1), and a constant term is added as necessary.

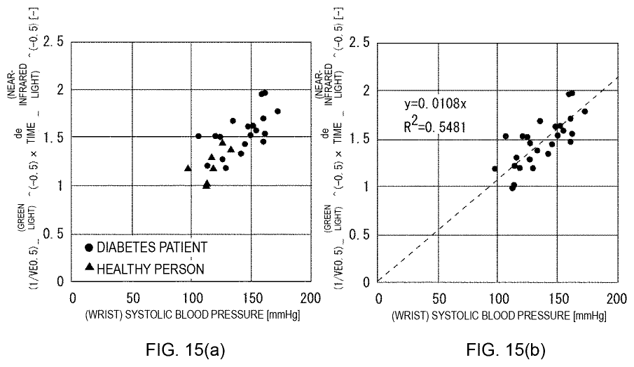

The graphs of FIG. 15 show an example of the relationship between the calculated value of Expression (1) and the (wrist) systolic blood pressure. In these graphs, the calculation was performed with the subscript a in Expression (1) being green light, the subscript b in the expression being near-infrared light, and the exponents α and β being 0.5. In addition, the wrist-type cuff sphygmomanometer was mounted on the left wrist (or the right wrist) of the user 40. The finger-mounting type sensing device 20 illustrated in FIG. 2 was attached to the index finger (or other fingers) of the left hand, the left hand on which the sensing device 20 was mounted is held at the chest height in the resting sitting position, and the photoplethysmographic wave and the blood pressure were measured.

The graph of FIG. 15(a) shows the distribution of the blood pressure index values for the diabetes patient and the healthy person. The graph of FIG. 15(b) shows the correlation between the calculated value of Expression (1) and the (wrist) systolic blood pressure. In each graph, the horizontal axis is the systolic blood pressure of the wrist, and the vertical axis is the calculated value of Expression (1). In the entire group of the diabetes patients and the healthy persons, on the assumption that the calculated value of Expression (1) is proportional to the systolic blood pressure, a linear approximation expression of the calculated value of Expression (1) and the systolic blood pressure was represented as y=0.0108x as illustrated in FIG. 15(b), and a determination coefficient R² of the approximation expression was determined to be about 0.55 (=0.5481). Since the correlation coefficient is a square root of the determination coefficient R², the correlation coefficient is 0.74, and it can be said that there is a strong correlation between the calculated value of Expression (1) and the systolic blood pressure.

As described in Expression (2) as follows, in the example of the blood pressure index-based expression (a: green light, b: near-infrared light, α=β=0.5), the blood pressure index-based expression is a reciprocal of a geometric mean of the pulse wave feature amount 1/VE0.5 (green light) of the photoplethysmographic signal measured with green light and the de time (near-infrared light) of the photoplethysmographic signal measured with near-infrared light.

$$1 / {\{ {( 1 / VE 0.5 )}_{G REEN LIGHT} \times de {TIME}_{N EAR - INFRARED LIGHT} \}}^{0.5} \qquad ( 2 )$$

It is estimated that the peripheral blood pressure index calculated from the pulse wave feature amount is reduced as the peripheral blood vessel function is deteriorated, but Expression (2) may show that the de time (near-infrared light) is increased as the peripheral blood vessel function is reduced. The number of pieces of data used for the calculation of the graphs of FIG. 15 is statistically insufficient, with 19 pieces of data of the diabetes patient data for 17 subjects, 7 pieces of data of the healthy person data for 7 subjects, that is 26 pieces of data for 24 subjects in total.

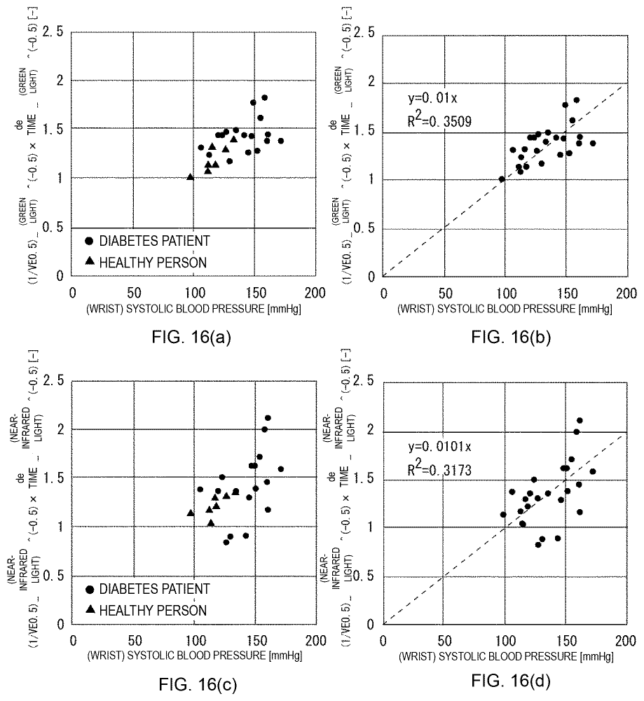

In addition, the graphs of FIGS. 16(a) and 16(b) show an example in which the calculation was performed with the subscripts a and b in Expression (1) being green light and the exponents α and β being 0.5. In these graph as well, in the entire group of the diabetes patients and the healthy persons, on the assumption that the calculated value of Expression (1) is proportional to the systolic blood pressure, a linear approximation expression of the calculated value of Expression (1) and the systolic blood pressure was represented as y=0.01x as illustrated in FIG. 16(b), and a determination coefficient R² of the approximation expression was determined to be about 0.35 (=0.3509).

In addition, the graphs of FIGS. 16(c) and 16(d) show an example in which the calculation was performed with the subscripts a and b in Expression (1) being near-infrared light and the exponents α and β being 0.5. In these graphs as well, in the entire group of the diabetes patients and the healthy persons, on the assumption that the calculated value of Expression (1) is proportional to the systolic blood pressure, a linear approximation expression of the calculated value of Expression (1) and the systolic blood pressure was represented as y=0.0101x as illustrated in FIG. 16(d), and a determination coefficient R² of the approximation expression was determined to be about 0.32 (=0.3173). In any of the approximation expressions in the graphs of FIGS. 16(b) and 16(d), the determination coefficient is reduced as compared with the approximation expression in the graph of FIG. 15(b). One of the reasons for the large variation in the diabetes patient data in the graph of FIG. 16(b) is that the variation in the de time (green light) calculated from the photoplethysmographic wave measured with the green light is large (see FIG. 12(e)). In addition, one of the reasons for the large variation in the diabetes patient data in the graph of FIG. 16(d) is that the variation in the pulse wave feature amount 1/VE0.5 (near-infrared light) calculated from the photoplethysmographic wave measured with the near-infrared light is large (see FIG. 10(b)).

The blood pressure index-based expression of Expression (2) was applied to data measured for a long period for the same subject. That is, the measurement of 24 sets was performed for 20 days, each set including data acquisition at the navel, the chest, and the forehead as measurement site heights. The measurement time zone was set to any of the morning, the daytime, and the evening, and 9 sets, 12 sets, and 3 sets of data were acquired, respectively.

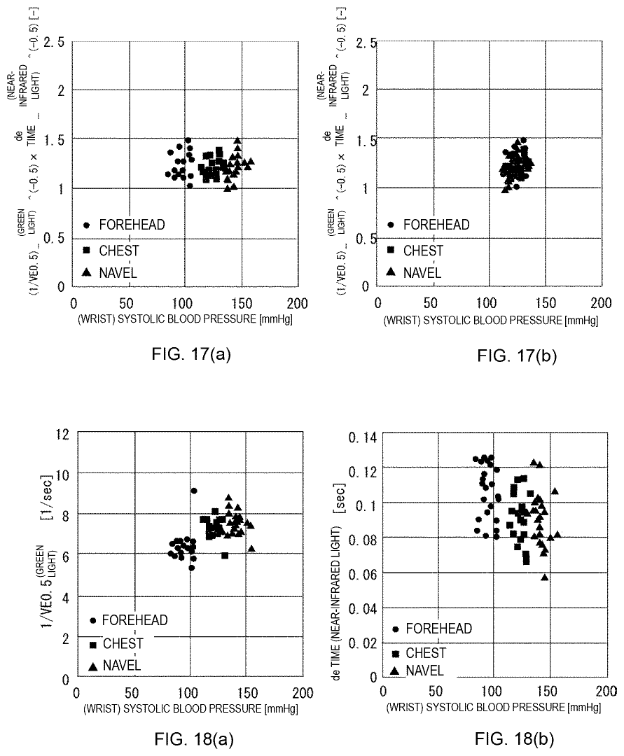

The graphs of FIGS. 17(a) and 17(b) show the relationship between the wrist systolic blood pressure and the blood pressure index value calculated from Expression (2), when the height of the measurement site (finger) from the heart is changed as described above. In each graph, the horizontal axis is the wrist systolic blood pressure, and the vertical axis is the blood pressure index value calculated from the blood pressure index-based expression of Expression (2). In addition, the triangular plot is a measurement result for the navel, the square plot is a measurement result for the chest, and the circular plot is a measurement result for the forehead.

The graph of FIG. 17(a) plots the blood pressure index value calculated from Expression (2) for the photoplethysmographic wave measured at each height with respect to each wrist systolic blood pressure measured at each height. The graph of FIG. 17(b) plots the blood pressure index value calculated from Expression (2) is plotted with respect to each wrist systolic blood pressure, which is obtained by replacing each systolic blood pressure measured at the height of the navel and the forehead with the measured value at the height of the chest, and unifying all the blood pressure index values at the height of the chest. The measurement order is photoplethysmographic wave (navel)→wrist blood pressure (navel)→photoplethysmographic wave (chest)→wrist blood pressure (chest)→photoplethysmographic wave (forehead)→wrist blood pressure (forehead), and the photoplethysmographic wave and the wrist blood pressure are not measured at the same time.

From the graph of FIG. 17(a), it is understood that the blood pressure index values at the respective heights of the navel, the chest, and the forehead are distributed in the substantially same range although there is variation. In addition, from the graph of FIG. 17(b), it is understood that, when all the blood pressure index values of the navel, the chest, and the forehead are unified to be measured at the height of the chest, the blood pressure index values at the respective heights of the navel, the chest, and the forehead substantially overlap.

FIG. 18 is a set of graphs in which a relationship with the systolic blood pressure is plotted for each of the pulse wave feature amounts 1/VE0.5 (green light) and de time (near-infrared light), which are the constituent elements of the blood pressure index-based expression. The graph of FIG. 18(a) shows the relationship between the pulse wave feature amount 1/VE0.5 (green light) and the systolic blood pressure, and the graph of FIG. 18(b) shows the relationship between the de time (near-infrared light) and the systolic blood pressure.

From the graph of FIG. 18(a), the pulse wave feature amount 1/VE0.5 (green light) tends to be large at the navel plotted in a triangular shape and be small at the forehead plotted in a circular shape. However, from the graph of FIG. 18(b), the de time (near-infrared light) shows an opposite tendency. The reason is that 1/VE0.5 (green light) has a positive correlation with the blood pressure as illustrated in the graph of FIG. 9(a) (in the case of a healthy person), and the de time (near-infrared light) has a negative correlation with the blood flow rate from the estimation of the above-described mechanism. Therefore, in the graphs of FIG. 17 illustrating the relationship between the blood pressure index value and the blood pressure, as a result, it is presumed that the reason why a significant change in the blood pressure index value was not observed even though the height of the measurement site from the heart was changed is that the changes in the pulse wave feature amounts 1/VE0.5 (green light) and de time (near-infrared light) were offset to some extent. Thus, by multiplying 1/VE0.5 (green light) by the de time (near-infrared light), the blood pressure index value estimated by Expression (2) can be presumed not to largely fluctuate even if the height of the measurement site is shifted from the height from the heart.

The blood pressure value at the height of the heart is useful for the user, and it is desired that the blood pressure value at the height of the heart can be estimated even if the height of the measurement site (finger) is shifted from the height of the heart from the viewpoint of usability. The above-described blood pressure index-based expression in the present disclosure is useful from the viewpoint of usability. However, in the diabetes patient in which the blood vessel function is deteriorated, there is a tendency that the change in 1/VE0.5 (green light) in response to the blood pressure change is small, and therefore, the above-described presumption that the respective pulse wave feature amounts are offset to some extent does not hold true. In the case of a user whose the peripheral blood vessel function is deteriorated, the measurement site needs to be held at the height of the heart.

In addition, when the blood pressure index/peripheral blood pressure index is defined as the blood pressure decrease index as an index indicating how much the blood pressure has decreased from the wrist to the capillary, the blood pressure decrease index can be represented by Expression (3).

$${( 1 / VE 0.5 )}_{a}^{- α - 1} \times de {TIME}_{b}^{- β} \qquad ( 3 )$$

Also here, the subscripts a and b represent the meaning of the green light or the near-infrared light, and represent the emission color of a measurement light source of the photoplethysmographic signal used for the calculation of the pulse wave feature amount 1/VE0.5 or de time. In addition, the exponents α and β indicating the power are positive numerical values. It is presumed that, as the value of the blood pressure decrease index calculated by Expression (3) becomes larger, the blood vessel resistance is increased, and the blood vessel disorder is more likely to occur.

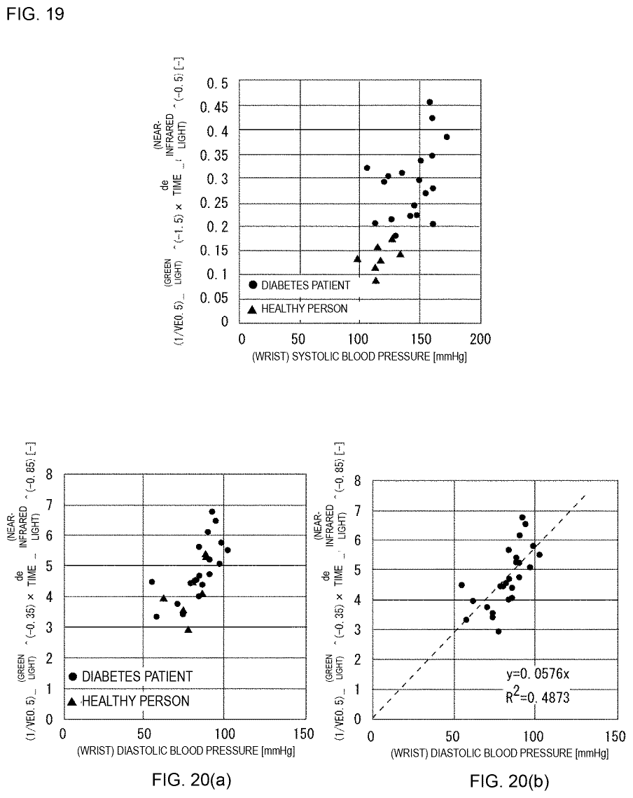

The graph of FIG. 19 shows an example of measuring the blood pressure decrease index and the blood pressure by calculating Expression (3) with the subscript a in Expression (3) being the green light, the subscript b in the expression being the near-infrared light, and the exponents a and B being 0.5. In the graph, the horizontal axis represents the systolic blood pressure of the wrist, and the vertical axis represents the magnitude of the blood pressure decrease index calculated by Expression (3). From the graph, it is understood that the blood pressure decrease index is increased in the diabetes patient.

If the actual measurement value of the peripheral blood pressure can be obtained, the actual decrease degree of the blood pressure can be calculated by multiplying the proportional coefficient by the blood pressure decrease index.

Although the systolic blood pressure has been described so far, the diastolic blood pressure can also be estimated by the similar method. A diastolic blood pressure index-based expression was created by using the feature amount used in the blood pressure index-based expression described in Expression (1).

FIG. 20 is a set of graphs illustrating an example of the relationship between the calculated value of the diastolic blood pressure index-based expression and the measured (wrist) diastolic blood pressure. In these graphs, the calculated values were calculated by using the diastolic blood pressure index-based expression with the subscript a in Expression (1) being the green light, the subscript b in the expression being the near-infrared light, and α=0.35 and β=0.85. In addition, the wrist-type cuff sphygmomanometer was mounted on the left wrist (or the right wrist) of the user 40. The finger-mounting type sensing device 20 illustrated in FIG. 2 was attached to the index finger (or other fingers) of the left hand, the left hand on which the sensing device 20 was mounted is held at the chest height in the resting sitting position, and the photoplethysmographic wave and the diastolic blood pressure were measured.

The graph of FIG. 20(a) shows the distribution of diastolic blood pressure index values for the diabetes patient and the healthy persons. The graph of FIG. 20(b) shows the correlation between the calculated value of the diastolic blood pressure index-based expression based on the above expression (1) and the (wrist) diastolic blood pressure. In each graph, the horizontal axis is the diastolic blood pressure of the wrist, and the vertical axis is the calculated value of the diastolic blood pressure index-based expression based on Expression (1). In the entire group of the diabetes patients and the healthy persons, on the assumption that the calculated value of the diastolic blood pressure index-based expression based on Expression (1) is proportional to the diastolic blood pressure, a linear approximation expression of the calculated value of the diastolic blood pressure index-based expression based on Expression (1) and the diastolic blood pressure was represented as y=0.0576x as illustrated in FIG. 20(b), and a determination coefficient R² of the approximation expression was determined to be about 0.49 (=0.4873). The correlation coefficient is 0.70, and it can be said that there is a strong correlation between the calculated value of the diastolic blood pressure index-based expression based on Expression (1) and the diastolic blood pressure.

When the diastolic blood pressure index-based expression is compared with the systolic blood pressure index-based expression, the absolute value of the power exponent of 1/VE0.5 (green light) is smaller and the absolute value of the power exponent of the de time (near-infrared light) is larger.

A blood pressure index-based expression in which the ae time is further added to Expression (1) is described in Expression (4) as follows.

$${( 1 / VE 0.5 )}_{a}^{- α} \times de {TIME}_{b}^{- β} \times ae {TIME}_{c}^{- γ} \qquad ( 4 )$$

Here, the subscripts a, b, and c represent the meaning of the green light or the near-infrared light, and represent the emission color of a measurement light source of the photoplethysmographic signal used for the calculation of the pulse wave feature amount 1/VE0.5, de time, or ae time. In addition, the exponents α, β, and γ indicating the power are positive numerical values.

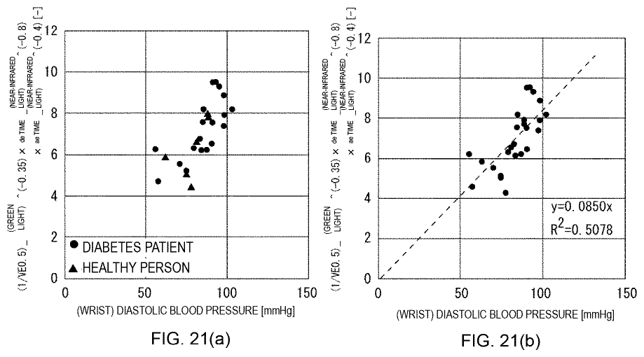

FIG. 21 is a graph illustrating an example of the relationship between the calculated value of the diastolic blood pressure index-based expression and the measured (wrist) diastolic blood pressure when calculation is performed by using the diastolic blood pressure index-based expression with the subscript a in Expression (4) being green light, the subscripts b and c in the expression being near-infrared light, and α=0.35, β=0.8, and γ=0.4. Each of the photoplethysmographic wave and the diastolic blood pressure was measured such that the wrist-type cuff sphygmomanometer was mounted on the left wrist (or the right wrist) of the user 40, the finger-mounting type sensing device 20 illustrated in FIG. 2 was attached to the index finger (or other fingers) of the left hand, and the left hand on which the sensing device 20 was mounted was held at the chest height in the resting sitting position.

The graph of FIG. 21(a) shows the distribution of diastolic blood pressure index values for the diabetes patient and the healthy persons. The graph of FIG. 21(b) shows the correlation between the calculated value of the diastolic blood pressure index-based expression based on Expression (4) and the (wrist) diastolic blood pressure. In each graph, the horizontal axis is the diastolic blood pressure of the wrist, and the vertical axis is the calculated value of the diastolic blood pressure index-based expression based on Expression (4). In the entire group of the diabetes patients and the healthy persons, on the assumption that the calculated value of the diastolic blood pressure index-based expression based on Expression (4) is proportional to the diastolic blood pressure, a linear approximation expression of the calculated value of the diastolic blood pressure index-based expression based on Expression (4) and the diastolic blood pressure was represented as y=0.0850x as illustrated in FIG. 21(b), and a determination coefficient R² of the approximation expression was determined to be about 0.51 (=0.5078). The correlation coefficient of the diastolic blood pressure index-based expression based on Expression (4) is 0.71, and the correlation coefficient is improved as compared with the correlation coefficient of the diastolic blood pressure index-based expression based on Expression (1).

In the diastolic blood pressure index-based expression based on Expression (4) used for creating the graph of FIG. 21, the ae time of the near-infrared light is used, but since there is no large difference in the ae time between the near-infrared light and the green light, the correlation coefficient is not largely affected even if the ae time of the green light is used.

It is said that the notch of the photoplethysmographic signal 53 in the recessed portion after the photoplethysmographic signal 53 has reached the maximal value corresponds to the end of the systolic period, and the e wave corresponds to the notch. Since there is no large difference in the ae time between the near-infrared light and the green light, it is presumed that the e-wave is less affected by the blood vessel state or the like. The fact that the ae time is long means that the time in which the left ventricle is contracted is long. Thus, it can be presumed that the ae time has a positive correlation with the stroke volume of one heartbeat. In addition, Expression (4) means that the diastolic blood pressure has a negative correlation with the ae time. Thus, it can be presumed that the diastolic blood pressure is decreased when the stroke volume of one heartbeat is increased.

It is said that, when the systolic blood pressure increases, a reflexive reaction occurs to open the peripheral blood vessels, the peripheral blood vessel resistance decreases, and the diastolic blood pressure is reduced. Since the systolic blood pressure increases when the stroke volume of one heartbeat increases, it is considered that the diastolic blood pressure is reduced by the above mechanism. Therefore, it is considered to be reasonable that the diastolic blood pressure has a negative correlation with the ae time as implied by Expression (4).

In a case where the calculation result of each of the diastolic blood pressure index-based expressions based on Expression (1) and Expression (4) is used as the blood pressure estimation value, a proportional coefficient is further multiplied by the diastolic blood pressure index value calculated by each of the diastolic blood pressure index-based expressions, and a constant term is added as necessary. In addition, the blood pressure index-based expression described in Expression (4) can also be used as the systolic blood pressure index-based expression by appropriately selecting the values of the exponents α, β, and γ indicating the powers.

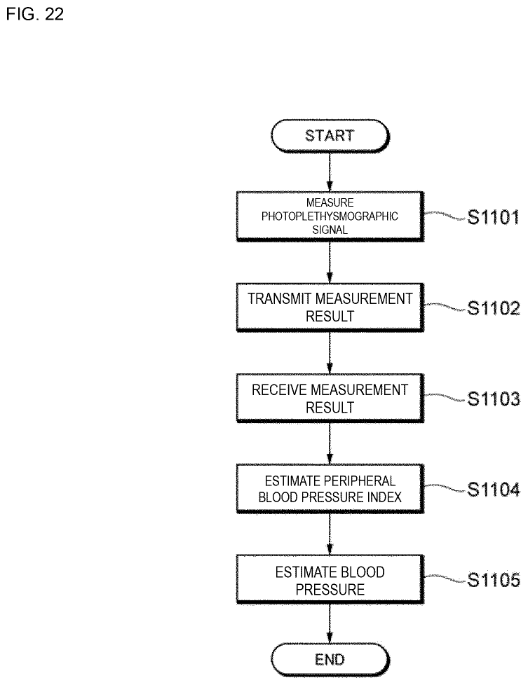

FIG. 22 is a flowchart illustrating an example of processing in the blood pressure estimation method according to aspects of the present disclosure. The processing by the biological information measurement system 10 is performed, for example, such that programs stored in non-transitory storage areas of the sensing device 20 and the computer 30 are executed by the sensing device 20 and the computer 30 including an information processing device such as a processor.

In Step S1101, the sensing device 20 of the biological information measurement system 10 measures a photoplethysmographic signal from the finger of a user who mounts the sensing device 20. Specifically, the photoplethysmographic sensor 211 measures a photoplethysmographic signal 53 by green light emitted by the green LED 211a and measures the photoplethysmographic signal 53 by near-infrared light emitted by the near-infrared LED 211b.

In Step S1102, the sensing device 20 transmits a measurement result to the computer 30 of the biological information measurement system 10. In Step S1103, the computer 30 receives the measurement result of the sensing device 20.

In Step S1104, the computer 30 calculates a peripheral blood pressure index of the user. For example, the computer 30 calculates pulse wave feature amounts 1/VE0.5, a/S, and (a-b)/(a-d) from the photoplethysmographic signal 53 measured by the biological sensor 21, and calculates the peripheral blood pressure index and the de time of the user from the calculated pulse wave feature amounts.

In Step S1105, the computer 30 calculates a blood pressure index value by using the above-described blood pressure index-based expression based on the peripheral blood pressure index and the de time stored in a storage unit such as the memory 322, and estimates the blood pressure of the user from the calculated blood pressure index value.

The exemplary aspect of the present disclosure has been described above. In the blood pressure estimation method described in the present aspect, the biological information measurement system 10 executes a step of acquiring the photoplethysmographic signal 53 of the blood vessel of the periphery of a user who is a subject by the photoplethysmographic sensor 211, a step of calculating the peripheral blood pressure index that is the index of the magnitude of the blood pressure of a capillary or an arteriole of the periphery based on the steepness of rising of the photoplethysmographic signal 53, and a step of estimating the magnitude of the blood pressure of the user by using the de time and the peripheral blood pressure index, the de time being a peak time difference between the d wave and the e wave in the acceleration pulse wave signal 52 obtained by performing second-order differentiation on the photoplethysmographic signal 53.

According to the present configuration, the photoplethysmographic signal 53 of the capillary or the arteriole of the periphery of the user is acquired by the photoplethysmographic sensor 211, and the peripheral blood pressure index that is the index of the magnitude of the blood pressure of the capillary or the arteriole of the periphery of the user is calculated based on the steepness of the rising of the acquired photoplethysmographic signal 53. The magnitude of the blood pressure of the user is estimated by using the calculated peripheral blood pressure index and the de time in the acceleration pulse wave signal 52 obtained by performing the second-order differentiation on the photoplethysmographic signal 53. Each of the peripheral blood pressure index and the de time used to estimate the blood pressure has a strong correlation with the blood pressure.

Therefore, according to the present configuration, the blood pressure estimation method is provided for estimating the blood pressure information of the user with high accuracy in a non-invasive manner.

In addition, in the blood pressure estimation method, the peripheral blood pressure index is calculated from the photoplethysmographic signal 53 acquired by the photoplethysmographic sensor 211 for at least the capillary of the periphery.

The peripheral blood pressure index has a stronger correlation with the blood pressure as the amount of information of the capillary is larger. Thus, according to the present configuration, the blood pressure of the user is estimated by using the peripheral blood pressure index having a stronger correlation with the blood pressure, so that blood pressure information of the user can be estimated with higher accuracy.

In addition, in the blood pressure estimation method, the de time is calculated from the photoplethysmographic signal 53 acquired by the photoplethysmographic sensor 211 for at least the peripheral arteriole.

The de time has a stronger correlation with the blood pressure as the amount of information of the arteriole is larger. Thus, according to the present configuration, the blood pressure of the user is estimated by using the de time having a stronger correlation with the blood pressure, so that blood pressure information of the user can be estimated with higher accuracy.

In addition, in the blood pressure estimation method, as described in Expression (1), the magnitude of the blood pressure of the user is estimated from the product of the power of the peripheral blood pressure index and the power of the de time.

According to the present configuration, the blood pressure of the user can be easily estimated by performing a calculation of a simple calculation expression.

In addition, in the blood pressure estimation method, the exponent of the power of the peripheral blood pressure index and the exponent of the power of the de time are negative values.

Both the peripheral blood pressure index and the de time have a strong negative correlation with the blood pressure. Thus, according to the present configuration, the blood pressure of the user can be estimated with high accuracy by performing the calculation with the exponents of the powers of the peripheral blood pressure index and the de time set to the negative values.

In addition, the blood pressure estimation method preferably includes a step of determining that the measurement site of the user whose photoplethysmographic signal 53 is measured by the photoplethysmographic sensor 211 is at the height of the heart.

The blood pressure changes depending on the height from the heart, but the blood pressure at the height of the heart is medically useful. Thus, with the present configuration, the blood pressure of the user can be estimated by using the photoplethysmographic signal 53 when the measurement site is at the height of the heart to perform the medical judgment, and the useful estimated blood pressure of the user can further be provided.

In addition, the blood pressure estimation method preferably includes a step of acquiring the height, from the heart, of the measurement site of the user whose photoplethysmographic signal 53 is measured by the photoplethysmographic sensor 211.

According to the present configuration, it is possible to determine at what height the measurement site is located with respect to the heart when the estimated blood pressure of the user is estimated by using the photoplethysmographic signal 53. Thus, in a case where the estimated blood pressure of the user is estimated by using the photoplethysmographic signal 53 when the measurement site is largely shifted from the height of the heart, it is possible to determine that the blood pressure estimation accuracy is low or not to output the blood pressure estimation value. In addition, the user can be notified that the measurement site is largely shifted from the height of the heart and to prompt the user to adjust the height of the measurement site.

As a method of estimating the height of the measurement site from the heart, a case where the computer 30 is a mobile control unit such as a multifunctional mobile phone terminal called a smartphone and includes an imaging device that images the user 40, a display device that displays an image captured by the imaging device, an inclination sensor that detects an inclination of the mobile control unit, and a control device that controls the imaging device, the display device, and the inclination sensor. If the height of the measurement site from the heart can be estimated, it is possible to determine that the measurement site is at the height of the heart.

First, a first method of estimating the height of the measurement site from the heart will be described with reference to an image of the user 40 captured by the imaging device.

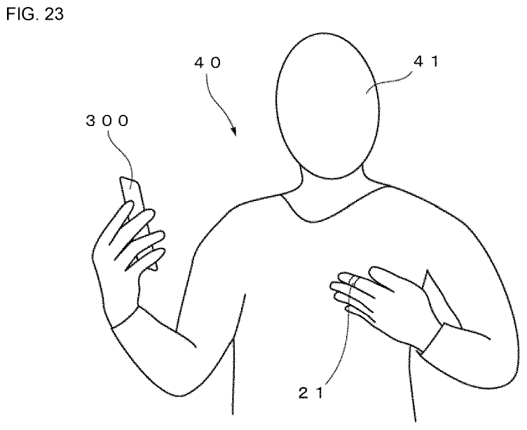

As illustrated in FIG. 23, the display device presents the user 40 who grips a mobile control unit 300 with one hand (for example, the right hand), with an instruction to move the other hand (for example, the left hand) on which the biological sensor 21 is mounted to a measurement position estimated to be at the height of the heart, and an instruction to image the other hand (for example, the left hand) on which the biological sensor 21 is mounted and the face 41 of the user 40 by the imaging device when the other hand (for example, the left hand) on which the biological sensor 21 is mounted is located at the measurement position. Then, the display device displays the image captured by the imaging device.

For example, a control device distinguishes and recognizes the face 41 of the user 40 and the other hand (for example, the left hand) on which the biological sensor 21 is mounted, from the image captured by the imaging device. The control device estimates a difference between the height of the heart and the height of the measurement site by comparing a relative positional relationship between the other hand (for example, the left hand) on which the biological sensor 21 is mounted and the face 41, which is obtained geometrically from the image captured by the imaging device, with a statistical positional relationship between the heart and the face 41. The control device outputs the estimation result of the difference between the height of the heart and the height of the measurement site to the signal processing device 32 as the height of the measurement site from the heart.

By estimating the statistical positional relationship between the hand and the face 41 of the user 40 from information indicating the physical features of the user 40 such as the height and the weight, the estimation accuracy can be improved of the height of the measurement site from the heart, from the relative positional relationship between the hand and the face 41 in the image.

In addition, for example, the control device distinguishes and recognizes the face 41 of the user 40 and the biological sensor 21 from the image captured by the imaging device. The control device estimates a difference between the height of the heart and the height of the measurement site, as the height of the measurement site from the heart, by comparing a relative positional relationship between the biological sensor 21 and the face 41, which is obtained geometrically from the image captured by the imaging device at the measurement position, with the statistical positional relationship between the heart and the face 41. The control device outputs the estimation result of the height of the measurement site from the heart to the signal processing device 32.

Next, a second method of estimating the height of the measurement site from the heart will be described with reference to an image of the user 40 captured by the imaging device.

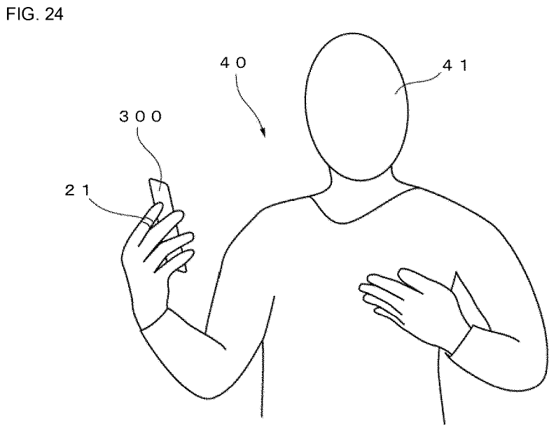

As illustrated in FIG. 24, the display device presents the user 40 who grips the mobile control unit 300 with the hand (for example, the right hand) on which the biological sensor 21 is mounted, with an instruction to move the hand (for example, the right hand) on which the biological sensor 21 is mounted to the measurement position estimated to be the height of the heart, and an instruction to image the face 41 of the user 40 by the imaging device when the hand (for example, the right hand) on which the biological sensor 21 is mounted is located at the measurement position. Then, the display device displays the image captured by the imaging device.

The control device estimates the difference between the height of the heart and the height of the measurement site by comparing a relative positional relationship between the face 41 of the user 40 and the mobile control unit 300, and a relative positional relationship between the heart and the face 41, which is geometrically obtained based on an inclination of the mobile control unit 300 with respect to a predetermined reference line (for example, a vertical line), with the statistical positional relationship between the heart and the face 41, from the image captured by the imaging device at the measurement position. The inclination sensor detects the inclination of the mobile control unit 300 with respect to the predetermined reference line (for example, a vertical line) when the user 40 changes the posture to measure the pulse wave signal at the measurement position.

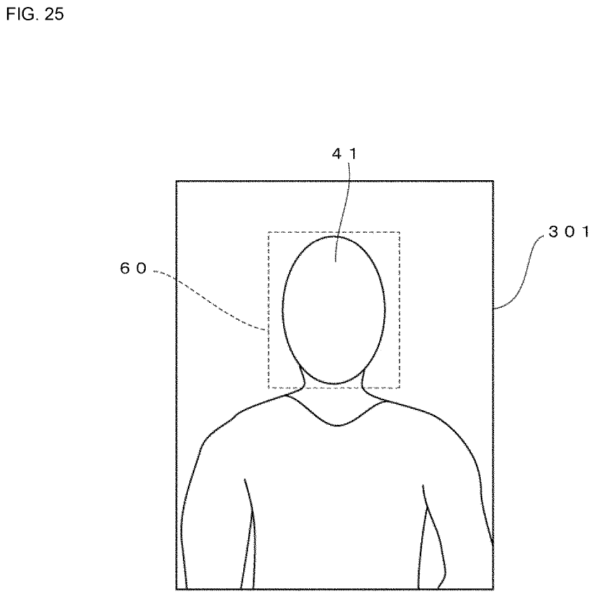

As illustrated in FIG. 25, the display device may graphically display a display target range 60 indicating a target position and a display target size of the face 41 to be superimposed on the face 41 displayed on a display device 301. By adjusting the positional relationship between the face 41 and the display device 301 such that the display position and the display size of the face 41 match the target position and the display target size of the face 41, respectively, it is possible to estimate the difference between the height of the heart and the height of the measurement site as the height of the heart from the measurement site based on the positional relationship between the face 41 of the user 40 and the mobile control unit 300 and the inclination of the mobile control unit 300 with respect to a predetermined reference line (for example, a vertical line) from the image captured by the imaging device. The control device outputs the estimation result of the height of the measurement site from the heart to the signal processing device 32.

By estimating the size (total head height, head width, and the like) of the face of the user 40 from the information indicating the physical features of the user 40 such as the height and the weight, the estimation accuracy can be improved of the height of the measurement site from the heart in the image, from the size of the face in the image.

In addition, the blood pressure estimation method preferably includes a step of correcting the blood pressure estimation value of the user based on the acquired height of the measurement site of the user from the heart.

According to the present configuration, the blood pressure estimation accuracy can be improved by correcting the blood pressure estimation value in a case where the measurement site is shifted from the height of the heart. In addition, since the blood pressure can be estimated by correcting the blood pressure estimation value of the user even if the measurement site is not held at the height of the heart, it is possible to continuously or intermittently perform the blood pressure estimation of the user.

The signal processing device 32 performs processing of correcting the blood pressure estimation value of the user in consideration of the influence of a hydrostatic pressure. In general, in a case where the measurement position of the blood pressure is higher than the heart, the measured value of the blood pressure is lowered only by a difference in hydrostatic pressure in the blood vessel due to gravity. On the contrary, in a case where the measurement position of the blood pressure is lower than the heart, the measured value of the blood pressure is increased only by the difference in hydrostatic pressure in the blood vessel.

The signal processing device 32 performs processing of calculating the pulse wave feature amount from the photoplethysmographic signal 53 measured by the photoplethysmographic sensor 211 and estimating the blood pressure from the pulse wave feature amount, for example, at each of at least two measurement positions at which the height from the heart of the user is different. A posture in which the user measures the photoplethysmographic signal 53 at at least two measurement positions at which the height of the heart of the user is different may be a posture of a sitting position or a posture of a supine position.

The signal processing device 32 obtains a correlation relationship between the blood pressure and the pulse wave feature amount of the user from the difference in height between at least two measurement positions at different heights from the heart of the user and the change in the pulse wave feature amount at the at least two measurement positions. For example, from the change in the pulse wave feature amount when the blood pressure changes from a low state to a high state (when the measurement position changes from a high state to a low state), the correlation relationship between the blood pressure and the pulse wave feature amount can be obtained.

In the processing of obtaining the correlation relationship between the blood pressure and the pulse wave feature amount of the user, it is sufficient that the tendency of the change in the pulse wave feature amount with respect to the change in the blood pressure is known. In a process of obtaining the tendency of the change in the pulse wave feature amount with respect to the change in the blood pressure, a difference in height between two measurement positions is obtained, and a difference in blood pressure corresponding to the difference in height is taken into consideration, so that it is possible to estimate the tendency of the change in the pulse wave feature amount with respect to the change in the blood pressure with high accuracy. The signal processing device 32 performs processing of correcting the blood pressure estimation value of the user from the pulse wave feature amount accurately calculated, based on this estimation.

In addition, in the blood pressure estimation method, it is preferable that the peripheral blood pressure index be calculated from the pulse wave feature amount represented by the reciprocal 1/VE0.5 of the width at a half value of the peak value of the waveform of the velocity pulse wave signal 51 obtained by performing first-order differentiation on the photoplethysmographic signal 53.

According to the present configuration, the peripheral blood pressure index is calculated based on the pulse wave feature amount 1/VE0.5, and is less likely to be affected by the noise or the individual difference in a photoplethysmographic waveform, and the blood pressure calculated by using the peripheral blood pressure index can be estimated with less influence of the noise or the individual difference for a wide range of users.

In addition, in the blood pressure estimation method, it is preferable that the peripheral blood pressure index be calculated from the pulse wave feature amount represented by the value a/S obtained by dividing the peak value a of the a wave of the acceleration pulse wave signal 52, which is obtained by performing second-order differentiation on the photoplethysmographic signal 53, by the maximum amplitude value S of the photoplethysmographic signal 53.

According to the present configuration, the peripheral blood pressure index is calculated based on the pulse wave feature amount a/S. Thus, the blood pressure of the user can be estimated by using the peripheral blood pressure index with the simple calculation method.

In addition, in the blood pressure estimation method, it is preferable that the peripheral blood pressure index be calculated from the pulse wave feature amount represented by the value calculated by the calculation expression (a-b)/(a-d) when the peak values of the a wave, the b wave, the c wave, and the d wave of the acceleration pulse wave signal 52 obtained by performing the second-order differentiation on the photoplethysmographic signal 53 are a, b, c, and d, respectively.

According to the present configuration, the peripheral blood pressure index is calculated based on the pulse wave feature amount (a-b)/(a-d). Thus, even with the present configuration, the blood pressure of the user can be estimated by using the peripheral blood pressure index with the simple calculation method.

In addition, in the blood pressure estimation method, it is preferable that the photoplethysmographic sensor 211 emit light in the wavelength band of blue to yellow-green from the first light source and emit light in the wavelength band of red to near-infrared from the second light source.

According to the present configuration, the light in the wavelength band from blue to yellow-green, which is strongly absorbed by the living body, is emitted from the first light source of the photoplethysmographic sensor 211 to the living body of the user. Thus, the photoplethysmographic signal 53 containing a large amount of information of the capillary in a shallow biological region from the skin surface of the living body is acquired by the photoplethysmographic sensor 211. In addition, the light having the wavelength band from red to near-infrared, in which the absorption of the living body is relatively small, is emitted from the second light source to the living body of the user. Thus, the photoplethysmographic signal 53 containing a large amount of information of the arteriole in the deep biological region from the skin surface of the living body is acquired by the photoplethysmographic sensor 211. Thus, the blood pressure of the user can be estimated with high accuracy by calculating the peripheral blood pressure index and the de time by using the photoplethysmographic signal 53 measured by the first light source and the photoplethysmographic signal 53 measured by the second light source.

In addition, in the blood pressure estimation method, it is preferable that, in the photoplethysmographic sensor 211, the distance between the first light source and a light receiving element that receives reflected light of the light emitted from the first light source be set to 1 to 3 mm, and the distance between the second light source and a light receiving element that receives reflected light of the light emitted from the second light source be set to 5 to 20 mm.

With the present configuration, since the distance between the first light source of the photoplethysmographic sensor 211 and the light receiving element is small, the photoplethysmographic signal 53 containing the more amount of information of the shallow biological region of the skin, that is, the information of the capillaries of the periphery is acquired. In addition, since the distance between the second light source and the light receiving element is large, the photoplethysmographic signal 53 containing the more amount of information of the deep biological region of the skin, that is, the information of the arteriole of the periphery is acquired. Thus, the blood pressure of the user can be estimated with higher accuracy by calculating the peripheral blood pressure index and the de time by using the photoplethysmographic signal 53 measured by the first light source and the photoplethysmographic signal 53 measured by the second light source.

In addition, in the blood pressure estimation method, it is preferable that the photoplethysmographic sensor 211 be mounted on the sensing device 20 to be mounted on the finger of the user.

With the present configuration, the photoplethysmographic sensor 211 mounted on the sensing device 20 can continuously or intermittently stably acquire the photoplethysmographic signal 53 from the finger of the user. Therefore, the blood pressure of the user can be stably estimated.

In addition, the blood pressure estimation method preferably further includes a step of determining a resting state of the user whose photoplethysmographic signal 53 is measured by the photoplethysmographic sensor 211.

When the measurement site is moved, the blood in the blood vessel is subjected to inertia, and thus the pulse waveform of the photoplethysmographic signal 53 measured by the photoplethysmographic sensor 211 also fluctuate. In addition, the blood pressure increases during exercise or the like, but the blood pressure at the resting is medically useful. In addition, when the measurement site is moved, a contact state between the photoplethysmographic sensor 211 and the skin of the user may change, and noise (body movement noise) may be generated when the contact state changes. Thus, with the present configuration, the blood pressure of the user can be estimated at the time of useful resting by determining the resting state of the user by using the acceleration sensor 24, the gyro sensor, or the like, and performing the blood pressure estimation only when the user is in the resting state.

In addition, in the blood pressure estimation method, the steps may be continuously or intermittently performed during the sleep of the user.

In healthy persons, the blood pressure is reduced during sleep and increases during wakefulness, but it is said that individuals in whom the blood pressure is not reduced or increases during sleep (nocturnal hypertension) have a high risk of cardiovascular diseases or cerebrovascular diseases. It is known that the risk of cerebrovascular disease is increased in a case where, with respect to a dipper type in which the blood pressure during sleep is lower than the blood pressure during wakefulness, the blood pressure during sleep is substantially same as the blood pressure during wakefulness (non-dipper type), the blood pressure during sleep increases (riser type), or the blood pressure during sleep is excessively low (extreme dipper type). With the present configuration, it is possible to detect the nocturnal hypertension by continuously or intermittently measuring the photoplethysmographic signal 53 during the sleep of the user to perform the blood pressure estimation of the user and ascertaining the change in the blood pressure estimation value.

In addition, the blood pressure estimation method may further include a step of determining whether or not the user is in a sleep state.

It is known that eating, drinking, caffeine intake, and smoking affect blood pressure. In addition, the blood pressure is also affected by exercise, walking, work using the body (cleaning and the like), bathing, conversation and mental tension, an environment with noise and vibration, and a cold environment. These events frequently occur at the time of wakefulness, and it is difficult to determine at which timing the events occur. On the other hand, during sleep, the influence of the event as described above can be reduced, and thus this time is suitable for stably measuring the blood pressure. With the present configuration, by providing the step of determining whether or not the user is sleeping from the activity level, the body surface temperature, the pulse rate, and the like, it is possible to easily determine whether or not the blood pressure of the user is increased during sleep. Thus, with the present configuration, the accuracy of the blood pressure estimation can be improved by distinguishing between the awake state and the sleep state of the user and performing the blood pressure estimation in the sleep state.

The determination of the sleep will be described. When the acceleration of the biological sensor 21 detected by the acceleration sensor 24 exceeds a predetermined value, it is determined that there is the body movement. When the number of body movements within a predetermined time is less than a threshold value, it is determined that the user is in sleep. The acceleration of the biological sensor 21 may suddenly increase due to the rolling over even during sleep, but the frequency thereof is lower than that during wakefulness. The finger has a higher frequency of movement during the wakefulness than the waist to which the activity meter is attached, a chest pocket, or other places such as a wrist. Therefore, a method of simply determining that the user is during the sleep in a case where the average value of the acceleration of the biological sensor 21 for a predetermined time is less than a threshold value may be used. In addition, the sleep determination accuracy may be improved by estimating the circadian rhythm from the body surface temperature of the finger by using the fact that the temperature of the finger rises during sleep and combining the estimation with the detection of the acceleration of the biological sensor 21. In addition, since the pulse rate is reduced during sleep and the respiratory rate is likely to be superimposed on the pulse rate, the sleep determination accuracy may be improved by adding the trend of the pulse rate.

In addition, the blood pressure estimation method may further include a step of estimating the degree of the decrease in the blood pressure of the capillary or the arteriole of the periphery from the blood pressure of the artery upstream of the arteriole, as the blood pressure decrease index obtained by dividing the blood pressure index of the artery by the peripheral blood pressure index 1/VE0.5 (the product of the power of 1/VE0.5 and the power of the de time) as in Expression (3).

With the present configuration, by estimating the blood pressure decrease index, it is possible to estimate how much the blood pressure of the periphery (capillary) is decreased from the blood pressure of the upper arm or the like. It can be estimated that the higher the value of the blood pressure decrease index, the higher the blood vessel resistance, and the blood vessel disorder has occurred.

In addition, in the blood pressure estimation method, the exponent of the power of 1/VE0.5 of the blood pressure decrease index and the exponent of the power of the de time are negative values.

Both the peripheral blood pressure index 1/VE0.5 and the de time have a strong negative correlation with the blood pressure. Thus, according to the present configuration, it is possible to easily estimate the blood pressure of the user with high accuracy by performing the calculation with the exponents of the powers of the peripheral blood pressure index 1/VE0.5 and the de time set to the negative values.

In general, the description of the aspects disclosed should be considered as being illustrative in all respects and not being restrictive. The scope of the present disclosure is shown by the claims rather than by the above description, and is intended to include meanings equivalent to the claims and all changes in the scope. While preferred aspects of the invention have been described above, it is to be understood that variations and modifications will be apparent to those skilled in the art without departing from the scope and spirit of the invention.

### DESCRIPTION OF REFERENCE SYMBOLS

10 biological information measurement system
20 sensing device
21 biological sensor
211 photoplethysmographic sensor
211a green led (first light source)
211b near-infrared led (second light source)
211c light receiving element
22 control circuit
23 communication module
24 acceleration sensor
25 housing
30 computer
31 communication module
32 signal processing device

1. A blood pressure estimation method comprising:
acquiring, by a biological information measurement system, a photoplethysmographic signal of a blood vessel of a periphery of a subject with a photoplethysmographic sensor;
calculating, by the biological information measurement system, a peripheral blood pressure index as an index of a magnitude of a blood pressure of a capillary or an arteriole of the periphery based on a steepness of rising of the photoplethysmographic signal; and
estimating, by the biological information measurement system, a magnitude of a blood pressure of the subject via a de time and the peripheral blood pressure index, the de time being a peak time difference between a d wave and an e wave in an acceleration pulse wave signal obtained by performing second-order differentiation on the photoplethysmographic signal.

2. The blood pressure estimation method according to claim 1, further comprising:
calculating the peripheral blood pressure index from the photoplethysmographic signal acquired by the photoplethysmographic sensor for at least the capillary of the periphery; and
calculating the de time based on the photoplethysmographic signal acquired by the photoplethysmographic sensor for at least the arteriole of the periphery.

3. The blood pressure estimation method according to claim 1, further comprising estimating the magnitude of the blood pressure of the subject from a power of the peripheral blood pressure index and a power of the de time.

4. The blood pressure estimation method according to claim 1, further comprising estimating the magnitude of the blood pressure of the subject based on using an ae time that is a peak time difference between an a wave and the e wave in the acceleration pulse wave signal obtained by performing the second-order differentiation on the photoplethysmographic signal.

5. The blood pressure estimation method according to claim 1, wherein the blood pressure of the subject is a systolic blood pressure.

6. The blood pressure estimation method according to claim 1, wherein the blood pressure of the subject is a diastolic blood pressure.

7. The blood pressure estimation method according to claim 1, further comprising:
acquiring a height, via a heart, of a measurement site of the subject whose photoplethysmographic signal is measured by the photoplethysmographic sensor; and
correcting a blood pressure estimation value of the subject based on the acquired height of the measurement site of the subject via the heart.

8. The blood pressure estimation method according to claim 1, wherein the peripheral blood pressure index includes at least one of information regarding a width of a peak which first appears within one beat of a waveform of a velocity pulse wave signal obtained by performing first-order differentiation on the photoplethysmographic signal, information regarding a peak value of an a wave of the acceleration pulse wave signal obtained by performing the second-order differentiation on the photoplethysmographic signal and a maximum amplitude value of the photoplethysmographic signal, or information regarding a peak difference (a-b) and a peak difference (a-d) when peak values of the a wave, a b wave, a c wave, and the d wave of the acceleration pulse wave signal obtained by performing the second-order differentiation on the photoplethysmographic signal are a, b, c, and d, respectively.

9. The blood pressure estimation method according to claim 1, further comprising controlling the photoplethysmographic sensor to emit light in a wavelength band from blue to yellow-green from a first light source, and to emit light in a wavelength band from red to near-infrared from a second light source.

10. The blood pressure estimation method according to claim 9, wherein, in the photoplethysmographic sensor, a distance between the first light source and a light receiving element that receives reflected light of the light emitted from the first light source is set to 1 to 3 mm, and a distance between the second light source and a light receiving element that receives reflected light of the light emitted from the second light source is set to 5 to 20 mm.

11. The blood pressure estimation method according to claim 1, wherein the photoplethysmographic sensor is mounted to a device configured to be worn on a finger of the subject.

12. The blood pressure estimation method according to claim 1, further comprising determining a resting state of the subject whose photoplethysmographic signal is measured by the photoplethysmographic sensor.

13. The blood pressure estimation method according to claim 1, further comprising performing the blood pressure estimation method continuously or intermittently during a sleep state of the subject.

14. The blood pressure estimation method according to claim 1, further comprising determining when the subject is in a sleep state.

15. The blood pressure estimation method according to claim 1, further comprising estimating a degree of a decrease in the blood pressure of the capillary or the arteriole of the periphery from a blood pressure of an artery upstream of the arteriole, as a blood pressure decrease index calculated from a blood pressure index of the artery and the peripheral blood pressure index.

16. The blood pressure estimation method according to claim 15, further comprising calculating the blood pressure decrease index based on a power of the peripheral blood pressure index and a power of the de time, and an exponent of each power is a negative value.

17. A biological information measurement system comprising:
a sensing device including a photoplethysmographic sensor configured to acquire a photoplethysmographic signal of a blood vessel of a periphery of a subject; and
a signal processing device configured to calculate a peripheral blood pressure index as an index of a magnitude of a blood pressure of a capillary or an arteriole of the periphery based on a steepness of rising of the photoplethysmographic signal, and to estimate a magnitude of a blood pressure of the subject by using a de time and the peripheral blood pressure index,
wherein the de time is a peak time difference between a d wave and an e wave in an acceleration pulse wave signal obtained by performing second-order differentiation on the photoplethysmographic signal.

18. The biological information measurement system according to claim 17, wherein the signal processing device is further configured to calculate the peripheral blood pressure index from the photoplethysmographic signal acquired by the photoplethysmographic sensor for at least the capillary of the periphery, and to calculate the de time from the photoplethysmographic signal acquired by the photoplethysmographic sensor for at least the arteriole of the periphery.

19. The biological information measurement system according to claim 17, wherein the signal processing device is further configured to estimate the magnitude of the blood pressure of the subject from a power of the peripheral blood pressure index and a power of the de time.

20. The biological information measurement system according to claim 17, wherein the signal processing device is further configured to estimate the magnitude of the blood pressure of the subject based on an ae time that is a peak time difference between an a wave and the e wave in the acceleration pulse wave signal obtained by performing the second-order differentiation on the photoplethysmographic signal.
